SEQUENCE LISTING (25 sequences)

SEQ ID NO: 1 - DNA, 5121 bp, unidentified
  source 1..5121: mol_type = unassigned DNA
  CDS 1..5121
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatatcgacatgttcgccattatgccgcatctgtccaacttccgcgactcggcgcgcagttcggtgaccagcggcgattcggtgaccgattacctggcgcgcacgcggcgggccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgagcggacgttcgaagttctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggccggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggggccccggtggcggtggtcacttccttgctgaccggggctctcaacggcatcctgcgcggcgtgcagcagcccatcatcgaaaagctggccaacgattacgctcgcaagatcgacgagctgggcgggccgcaagcgtacttcgagaaaaacctgcaggcgcgtcacgaacaactggccaattcggacggcctacggaaaatgctggccgacctgcaggccggttggaacgccagcagcgtgatcggggtgcagacgacagagatctccaagtcggcgctcgaactggccgccattaccggcaacgcggacaacctgaaatccgtcgacgtgttcgtggaccgcttcgtccagggcgagcgggtggccggccagccggtggtcctcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggatgtgatcggcggagatggcgatgacgtcgtgcttgccaatgcttcgcgcatccattatgacggcggcgcgggcaccaacacggtcagctatgccgccctgggtcgacaggattccattaccgtgtccgccgacggggaacgtttcaacgtgcgcaagcagttgaacaacgccaacgtgtatcgcgaaggcgtggctacccagacaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctagccggcgggtcgggcgacgacaggctggatggcggcgccggcaacgacaccctggttggcggcgagggccaaaacacggtcatcggcggcgccggcgacgacgtattcctgcaggacctgggggtatggagcaaccagctcgatggcggcgcgggcgtcgataccgtgaagtacaacgtgcaccagccttccgaggagcgcctcgaacgcatgggcgacacgggcatccatgccgatcttcaaaagggcacggtcgagaagtggccggccctgaacctgttcagcgtcgaccatgtcaagaatatcgagaatctgcacggctcccgcctgaacgaccgcatcgccggcgacgaccaggacaacgagctctggggccacgatggcaacgacacgatacgcggccggggcggcgacgacatcctgcgcggcggcctgggcctggacacgctgtatggcgaggacggcaacgacatcttcctgcaggacgacgagaccgtcagcgatgacatcgacggcggcgcggggctggacaccgtcgactactccgccatgatccatccaggcaggatcgttgcgccgcatgaatacggcttcgggatcgaggcggacctgtccagggaatgggtgcgcaaggcgtccgcgctgggcgtggactattacgataatgtccgcaatgtcgaaaacgtcatcggtacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggcgacgccggcaacgacaccctctacggggggctgggcgacgatacccttgaaggcggcgcgggcaacgattggttcggccagacgcaggcgcgcgagcatgacgtgctgcgcggcggagatggggtggataccgtcgattacagccagaccggcgcgcatgccggcattgccgcgggtcgcatcgggctgggcatcctggctgacctgggcgccggccgcgtcgacaagctgggcgaggccggcagcagcgcctacgatacggtttccggtatcgagaacgtggtgggcacggaactggccgaccgcatcacgggcgatgcgcaggccaacgtgctgcgcggcgcgggtggcgccgacgtgcttgcgggcggcgagggcgacgatgtgctgctgggcggcgacggcgacgaccagctgtcgggcgacgccggacgcgatcgcttgtacggcgaagccggtgacgactggttcttccaggatgccgccaatgccggcaatctgctcgacggcggcgacggccgcgataccgtggatttcagcggcccgggccggggcctcgacgccggcgcaaagggcgtattcctgagcttgggcaaggggttcgccagcctgatggacgaacccgaaaccagcaacgtgttgcgcaatatcgagaacgccgtgggcagcgcgcgtgatgacgtgctgatcggcgacgcaggcgccaacgtcctcaatggcctggcgggcaacgacgtgctgtccggcggcgctggcgacgatgtgctgctgggcgacgagggctcggacctgctcagcggcgatgcgggcaacgacgatctgttcggcgggcagggcgatgatacttatctgttcggggtcgggtacgggcacgacacgatctacgaatcgggcggcggccatgacaccatccgcatcaacgcgggggcggaccagctgtggttcgcgcgccagggcaacgacctggagatccgcattctcggcaccgacgatgcacttaccgtgcacgactggtatcgcgacgccgatcaccgggtggaaatcatccatgccgccaaccaggcggtagaccaggcaggcatcgaaaagctggtcgaggcaatggcgcagtatccggaccccggcgcggcggcggctgccccgccggcggcgcgcgtgccggacacgctgatgcagtccctggctgtcaactggcgctga

SEQ ID NO: 2 - protein, 1706 aa, unidentified
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 3 - DNA, 5121 bp, unidentified; identical to SEQ ID NO: 1
  source 1..5121: mol_type = unassigned DNA
  CDS 1..5121

SEQ ID NO: 4 - protein, 1706 aa, unidentified; identical to SEQ ID NO: 2

SEQ ID NO: 5 - DNA, 5121 bp, unidentified
  source 1..5121: mol_type = unassigned DNA
  CDS 1..5121
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatatcgacatgttcgccatcatgccgcatctgtccaacttccgcgactcggcgcgcagttcggtgaccagcggcgattcggtgaccgattacctggcgcgcacgcggcgggccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgccgtcggcgcgcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcatacggcgtggcggggaaaagcctgttcgacgatgggctgggagccgcgcccggcgtgccgggcggacgttcgaagtcctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcgccgcggccgttggcgccgggatgtcgttgaccgatgacgcgccggccggacagaaggccgccgtcggcgccgagatcgcgctgcagttgacaggtggaacggtcgagctggcttcttccatcgcgttggcgctggccgcggcgcgcggcgtgaccagcggcttgcaggtggcgggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggcggcggtgtcgatcgccgcggcggccagcgtggtaggcgccccggtggcggtggtcacttccttgttgaccggggctctcaacggcatcctgcgcggcgtgcagcagcccatcatcgaaaagctggccaatgattacgctcgcaagatcgacgagctgggcgggccgcaagcgtacttcgagaaaaacctgcaggcgcgtcacgaacaactggccaattcggacggcctacggaaaatgctggctgacctgcaggccgggtggaacgccagcagcgtgatcggggtgcagacgacagagatttccaagtcggcgctcgaactggccgccattaccggcaacgcggacaacctgaaatccgccgacgtgttcgtggaccgcttcatccagggcgagcgggtggccggccagccggtggtactcgacgtcgccgccggcggcatcgatatcgccagccgcaagggcgagcggccggcgctgacgttcatcacgccgctggccgcgccaggagaagagcagcgccggcgcacgaagacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggaggtgatcggcggagatggcgatgatgtcgtgcttgccaatgcttcgcgcatccattacgacggcggcgcgggaaccaacacggtcagctatgccgccctgggccgacaggattccattaccgtgtccgccgacggggaacgtttcaacgtgcgcaagcagttgaacaacgccaacgtgtatcgcgaaggcgtggctacccagaaaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctggccggcggggcgggcgacgacaggctggatggcggcgccggcaacgacacactggtcggcggcgagggccacaacacggtcgtcggcggcgctggcgacgacgtattcctgcaggacctgggggtatggagcaaccagctcgatggcggcgcgggcgtcgataccgtgaagtacaacgtgcaccagccttccgaggaacgcctcgaacgcatgggcgacacgggcatccatgccgatcttcaaaagggcacggtcgagaagtggccggccctgaacctgttcagcgtcgaccatgtcaagaatatcgagaatctgcacggctccagcctgaacgacagcatcgccggcgacgaccgggacaacgagctctggggcgacgatggcaacgacacgatacacggccggggcggcgacgatatcctgcgcggcggcctgggcctggacacgctgtatggcgaggacggcaacgacatcttcctgcaggacgacgagaccgtcagcgatgacatcgacggcggcgcggggctggacaccgtcgactattccgccatgatccatgcaggcaagatcgttgcgccgcatgaatacggcttcgggatcgaggcggacctgtccgaagggtgggtgcgcaaggcggcccggcgcggcatgggctactacgacagtgtccgcagtgtcgaaaacgtcatcggcacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggagacgccggcaacgacaccctctacggagggctgggcgacgatacccttgaaggcggcgcgggcaacgattggttcggccagacgccggcgcgcgagcatgacgtgctgcgcggcggggctggggtggataccgtggattacagccaggcgggcgcgcatgccggcgttgccacgggtcgcatcgggctgggtattctggcggacctgggcgccggccgcgtcgacaagctgggcgaggccggcagcagcgcctacgatacggtttccggcatcgaaaatgtggtgggcacggaactggccgaccgcatcacgggcgatgcgcaggccaacgtactgcgcggcgcgggtggtgccgacgtgcttgcgggcggcgagggcgacgatgtgctgctgggcggcgagggcgatgaccagctgtcgggcgacgccggacgcgaccgcttgtacggcgaagccggtgacgactggttcttccaggatgccgccaatgccggcaatctgctcgacggtggtgacggcaacgataccgtggatttcagcggcccgggccggggcctcgacgccggcgcaaagggcgtattcctgagcctgggcaaggggttcgccagcctgatggacgaacccgaaaccagcaacgtgttgcgccatatcgagaacgccgtgggcagcgtgcgtgatgacgtgctgatcggcgacgcaggcgccaacgtcctcaatggcctggcgggcaacgacgtgttgtccggcggcgccggcgacgatgtgctgctgggcgacgagggctcggacctgctcagcggcgatgcgggcaacgacgatctgttcggcgggcagggcgatgatacctatctgttcggggccgggtacggacatgacacgatctacgaatcgggcggcggccatgacaccatccgtatcaacgcgggggcggaccagctgtggtttgcgcgccagggcaacgacctggagatccgcattcttggcaccgacgatgcacttaccgtgcacgactggtatcgcgacgccgatcaccgggtggaagccatccatgccgccaaccaggccatagacccggccggcatcgaaaagctggtcgaggcaatggcgcagtacccggaccccggcgcggcggcggctgccccgccggcggcgcgcgtgccggacacgctgatgcagtccctggctgtcaactggcgctga

SEQ ID NO: 6 - protein, 1706 aa, unidentified
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGAQDVVQHGTEQNNPFPEADEKIFVVSATGESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAVGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 7 - DNA, 5118 bp, unidentified
  source 1..5118: mol_type = unassigned DNA
  CDS 1..5118
atgcagcaatcgcatcaggctggttacgcaaacgccgccgaccgggagtctggcatccccgcagccgtactcgatggcatcaaggccgtggcgaaggaaaaaaacgccacattgatgttccgcctggtcaacccccattccaccagcctgattgccgaaggggtggccaccaaaggattgggcgtgcacgccaagtcgtccgattgggggttgcaggcgggctacattcccgtcaacccgaatctttccaaactgttcggccgtgcgcccgaggtgatcgcgcgggccgacaacgacgtcaacagcagcctggcgcatggccataccgcggtcgacctgacgctgtcgaaagagcggcttgactatctgcggcaagcgggcctggtcaccggcatggccgatggcgtggtcgcgagcaaccacgcaggctacgagcagttcgagtttcgcgtgaaggaaacctcggacgggcgctatgccgtgcagtatcgccgcaagggcggcgacgatttcgaggcggtcaaggtgatcggcaatgccgccggtattccactgacggcggatatcgacatgttcgccatcatgccgcatctgtccaacttccgcgactcggcgcgcagttcggtgaccagcggcgattcggtgaccgattacctggcgcgcacgcggcgggccgccagcgaggccacgggcggcctggatcgcgaacgcatcgacttgttgtggaaaatcgctcgcgccggcgcccgttccgcagtgggcaccgaggcgcgtcgccagttccgctacgacggcgacatgaatatcggcgtgatcaccgatttcgagctggaagtgcgcaatgcgctgaacaggcgggcgcacgcggtcggcaggcaggacgtggtccagcatggcactgagcagaacaatcctttcccggaggcagatgagaagattttcgtcgtatcggccaccggtgaaagccagatgctcacgcgcgggcaactgaaggaatacattggccagcagcgcggcgagggctatgtcttctacgagaaccgtgcgtacggcgtggcggggaaaagcctgttcgacgatgggctgggagccgcgcccggcgtgccggggcgacgttcgaagtcctcgccggatgtactggaaacggtgccggcgtcacccggattgcggcggccgtcgctgggcgcagtggaacgccaggattccggctatgacagccttgatggggtgggatcgcgatcgttctcgttgggcgaggtgtccgacatggccgccgtggaagcggcggaactggaaatgacccggcaagtcttgcacgccggggcgcggcaggacgatgccgagccgggcgtgagcggtgcgtcggcgcactgggggcagcgggcgctgcagggcgcccaggcggtggcggcggcgcagcggctggttcatgccattgccctgatgacgcaattcggccgggccggttccaccaacacgccgcaggaagcggcctcgttgtcggcggccgtgttcggcttgggcgaggccagcagcgccgtggccgaaaccgtgagcggttttttccgcgggtcttcgcgctgggccggcggtttcggcgtggctggcggcgcgatggcgctgggaggcggcatcggcgccgttggcgccgggatgtcgttgaccgatgacgcgccggccggacagaaggccgccgccggcgccgagatcgcgctgcagttgacaggtggaacggtcgagctggcttcttccatcgcgttggcgctggccgcggcgcgcggcgtgaccagcggcttgcaggtggcgggggcgtcggccggggcggctgccggcgcattggccgcggcgctcagtcccatggagatctacggcctggtgcagcaatcgcactatgcggatcagctggacaagctggcgcaggaatcgagcgcatacggttacgagggcgacgccttgctggcccagctgtatcgcgacaagacggccgccgagggcgccgtcgccggcgtctccgccgtcctgagcacggtgggggctgcggtgtcgatcgccgcggcggccagcgtggtaggcgccccggtggcggtggtcacttccttgttgaccggggctctcaacggcatcctgcgcggcgtgcagcagcccatcatcgaaaagctggccaatgattacgctcgcaagatcgacgagctgggcgggccgcaagcgtacttcgagaaaaacctgcaggcgcgtcacgaacaactggccaattcggacggcctacggaaaatgctggccgacctgcaggccgggtggaacgccagcagcgtgatcggggtgcagacgacagagatttccaagtcggcgctcgaactggccgccattaccggcaacgcggacaacctgaaatccgccgacgtgttcgtggaccgcttcatccagggcgagcgggtggccggccagccggtggtactcgacgtcgccgccggcggcatcgatatcgccagccgcaagggcgagcggccggcgctgacgttcatcacgccgctggccgcgccaggagaagagcagcgccggcgcacgaaaacgggcaagagcgaattcaccacattcgtcgagatcgtgggcaagcaggaccgctggcgcatccgggacggcgcggccgacaccaccatcgatctggccaaggtggtgtcgcaactggtcgacgccaatggcgtgctcaagcacagcatcaaactggaggtgatcggcggagatggcgatgatgtcgtgcttgccaatgcttcgcgcatccattacgacggcggcgcgggaaccaacacggtcagctatgccgccctgggccgacaggattccattaccgtgtccgccgacggggaacgtttcaacgtgcgcaagcagttgaacaacgccaacgtgtatcgcgaaggcgtggctacccagaaaaccgcctacggcaagcgcacggagaatgtccaataccgccatgtcgagctggcccgtgtcgggcaactggtggaggtcgacacgctcgagcatgtgcagcacatcatcggcggggccggcaacgattcgatcaccggcaatgcgcacgacaacttcctggccggcggggcgggcgacgacaggctggatggcggcgccggcaacgacacactggtcggcggcgagggccacaacacggtcgtcggcggcgctggcgacgacgtattcctgcaggacctgggggtatggagcaaccagctcgatggcggcgcgggcgtcgataccgtgaagtacaacgtgcaccagccttccgaggaacgcctcgaacgcatgggcgacacgggcatccatgccgatcttcaaaagggcacggtcgagaagtggccggccctgaacctgttcagcgtcgaccatgtcaagaatatcgagaatctgcacggctccagcctgaacgacagcatcgccggcgacgaccgggacaacgagctctggggcgacgatggcaacgacacgatacacggccggggcggcgacgatatcctgcgcggcggcctgggcctggacacgctgtatggcgaggacggcaacgacatcttcctgcaggacgacgagaccgtcagcgatgacatcgacggtggcgcgggactggacaccgtcgactattccgccatgatccatgcaggcaagatcgttgcgccgcatgaatacggcttcgggatcgaggcggacctgtccgaagggtgggtgcgcaaggcggcccggcgcggcatggactactacgacagtgtccgcagtgtcgaaaacgtcatcggcacgagcatgaaggatgtgctcatcggcgacgcgcaagccaataccctgatgggccagggcggcgacgataccgtgcgcggcggcgacggcgatgatctgctgttcggcggcgacggcaacgacatgctgtatggagacgccggcaacgacaccctctacggagggctgggcgacgatacccttgaaggcggcgcgggcaacgattggttcggccagacgccggcgcgcgagcatgacgtgctgcgcggcggggctggggtggataccgtggattacagccaggcgggcgcgcatgccggcgttgccacgggtcgcatcgggctgggtattctggcggacctgggcgccggccgcgtcgacaagctgggcgaggccggcagcagcgcctacgatacggtttccggcatcgaaaatgtggtgggcacggaactggccgaccgcatcacgggcgatgcgcaggccaacgtactgcgcggcgcgggtggcgccgacgtgcttgcgggcggcgagggcgacgatgtgctgctgggcggcgacggcgacgaccagctgtcgggcgacgccggacgcgaccgcttgtacggcgaagccggtgacgactggttcttccaggatgccgccaatgccggcaatctgctcgacggtggtgacggcaacgataccgtggatttcagcggcccgggccggggcctcgacgccggcgcaaagggcgtattcctgagcctgggcaaggggttcgccagcctgatggacgaacccgaaaccagcaacgtgttgcgccatatcgagaacgccgtgggcagcgtgcgtgatgacgtgctgatcggcgacgcaggcgccaacgtcctcaatggcctggcgggcaacgacgtgctgtccggcggcgccggcgacgatgtgctgctgggcgacgagggctcggacctgctcagcggcgatgcgggcaacgacgatctgttcggcgggcagggcgatgatacctatctgttcggggccgggtacggacatgacacgatctacgaatcgggcggcggccatgacaccatccgtatcaacgcgggggcggaccagctgtggtttgcgcgccagggcaacgacctggagatccgcattcttggcaccgacgatgcacttaccgtgcacgactggtatcgcgacgccgatcaccgggtggaagccatccatgccgccaaccaggccatagacccggccggcatcgaaaagctggtcgaggcaatggcgcagtacccggaccccggcgcggcggcggctgccccgccggcggcgcgcgtgccggacacgctgatgcagtccctggctgtcaactggcgctga

SEQ ID NO: 8 - protein, 1705 aa, unidentified
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASEATGGLDRERIDLLWKIARAGARSAVGTEARRQFRYDGDMNIGVITDFELEVRNALNRRAHAVGRQDVVQHGTEQNNPFPEADEKIFVVSATGESQMLTRGQLKEYIGQQRGEGYVFYENRAYGVAGKSLFDDGLGAAPGVPGRRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIGAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 9 - DNA, 4842 bp, synthetic construct
  misc_feature 1..4842: note = Nucleotide sequence encoding polypeptide 1-227 + 321-1706 of SEQ ID NO:2
  source 1..4842: mol_type = other DNA
  CDS 1..4842
atgcagcagtcccatcaggccggttacgcaaacgcagccgatcgtgaatctggtatcccggcagcggttctggatggtattaaggcagttgccaaagagaagaacgctaccctgatgttccgtctggtcaacccacactctacttccctgatcgcggaaggtgtggcgaccaaaggcctgggtgtgcacgccaaaagcagcgactggggtctgcaggcaggttatattccggtgaacccgaatctgtctaaactgttcggtcgtgccccggaggtgatcgcacgtgcagacaacgatgtcaactcttctctggcgcatggtcacaccgcggttgatctgaccctgtccaaagaacgcctggattacctgcgtcaggcgggcctggtgacgggcatggccgacggtgttgtagcgagcaaccacgcgggttatgaacaatttgagtttcgcgttaaagagacctctgacggtcgttacgcggtgcagtaccgccgcaaaggtggcgatgactttgaagctgtcaaagtgatcggtaacgctgctggaattccgctgaccgcggatatcgacatgtttgccatcatgcctcacctgtccaactttcgcgattccgcacgcagctctgtgacgtctggtgattccgtcactgactacctggctcgtacccgtcgtgcggcctctgaatcccagatgctgactcgtggccagctgaaagaatacatcggccaacagcgtggcgaaggctacgttttctacgagaatcgtgcttacggcgttgcgggcaaatccctgttcgacgatggtctgggcgctgcaccgggcgttccgagcggccgttctaaattcagcccggacgtactggaaactgtgccggcttccccgggcctgcgccgtccgtccctgggcgcagtcgaacgtcaggactccggctacgattccctggatggcgttggctcccgctccttctccctgggcgaggtttctgacatggctgcggtggaagcggcagagctggaaatgacccgtcaagtgctgcacgcgggcgcacgtcaggacgatgctgagccgggcgtgtccggtgcgtctgcacactggggtcaacgtgccctgcagggtgctcaagctgtcgcggcagcgcagcgtctggtacatgcgatcgcactgatgacccagttcggtcgcgcaggttctaccaacactcctcaggaagcagctagcctgtctgctgctgttttcggtctgggcgaagcgtcttctgcggttgcagaaaccgtgagcggtttcttccgtggttctagccgctgggcgggtggttttggtgtagcaggtggcgctatggccctgggtggcggtatcgcggctgctgtcggcgcaggtatgagcctgaccgacgatgctcctgcgggccagaaagccgcagctggcgctgaaatcgcgctgcagctgactggtggtaccgttgaactggctagctctatcgcgctggctctggcagcggcacgtggcgtgacttctggcctgcaagtcgccggtgcctctgcgggcgctgctgcgggcgctctggcagctgcactgtcccccatggaaatctacggtctggtacagcagtctcactacgcagaccagctggataaactggcgcaggagtcttctgcatacggttacgaaggcgacgcactgctggcgcagctgtatcgtgacaaaactgctgctgaaggtgctgtggcaggcgtttctgcggtactgtctaccgttggcgctgcggtttctattgcggctgccgcatccgttgtaggtgcgccggttgctgtagttacctctctgctgactggtgcactgaacggtatcctgcgtggtgttcagcagcctattatcgaaaaactggcgaatgattatgcccgtaaaatcgacgaactgggcggtccgcaggcttattttgaaaaaaacctgcaggcgcgccacgaacagctggcaaacagcgacggcctgcgcaaaatgctggcagacctgcaagctggttggaacgcgtcttctgtgatcggcgtgcagaccaccgaaattagcaaatctgcgctggaactggctgcaattactggcaacgcggataacctgaaaagcgttgatgtttttgtcgatcgcttcgttcagggcgagcgcgttgctggtcagccggttgttctggacgttgcggcaggcggcatcgacatcgctagccgcaagggcgagcgtccggctctgactttcattaccccgctggcagctccgggtgaagaacagcgtcgccgtaccaagactggtaaaagcgagttcaccaccttcgtggaaatcgtaggtaaacaggaccgctggcgtattcgcgacggtgcggcggacaccaccatcgacctggcaaaagttgtttcccaactggtagatgctaacggcgtgctgaaacattctattaaactggatgtaatcggcggtgacggcgatgacgttgtactggcgaacgcgtctcgtatccactatgacggcggcgcaggtaccaacacggtttcctacgcagcgctgggccgccaggattccatcactgttagcgccgatggcgaacgtttcaacgtgcgtaaacagctgaataacgcaaacgtctatcgtgagggtgtagctacccagaccaccgcgtacggtaagcgtaccgaaaacgttcagtatcgtcacgtggaactggcacgtgtgggtcagctggtagaggtggatacgctggaacacgtgcagcacatcatcggtggcgccggtaacgattccattactggtaacgctcacgacaacttcctggcaggtggttctggtgatgatcgtctggacggtggtgcgggtaacgatacgctggtaggcggcgaaggtcagaacaccgtaattggtggtgccggtgacgacgttttcctgcaggatctgggcgtttggagcaaccagctggatggcggtgccggtgttgacacggttaagtataacgttcaccagccgtctgaagagcgcctggaacgtatgggtgacactggtattcatgccgatctgcagaaaggcactgttgaaaaatggcctgcgctgaacctgttctctgtggaccatgttaagaacattgaaaacctgcatggctctcgcctgaacgaccgtatcgctggtgacgaccaggataacgaactgtggggtcatgacggcaatgataccattcgcggtcgtggcggcgacgatattctgcgtggcggtctgggcctggataccctgtatggtgaagatggtaacgatattttcctgcaagacgatgaaacggtatctgatgacatcgacggtggtgcaggcctggacaccgtagattacagcgctatgatccatccgggtcgtatcgtagctccgcacgagtacggcttcggtatcgaggcggacctgtctcgtgaatgggttcgtaaagcgtccgcgctgggtgtggactactacgataacgttcgtaacgtggaaaacgtgatcggtacctccatgaaagacgtgctgattggcgacgcacaggccaacactctgatgggccagggcggtgatgacacggttcgcggcggcgacggtgatgacctgctgttcggtggtgacggcaacgacatgctgtacggcgacgcgggcaacgacaccctgtacggcggtctgggtgacgacactctggaaggtggtgcaggtaacgactggttcggccaaacccaggcacgcgaacacgacgtgctgcgtggtggcgacggcgtagacaccgtggattactcccaaactggcgctcacgcgggtatcgcggccggtcgtatcggtctgggcattctggccgatctgggtgccggccgtgtcgacaaactgggtgaggctggctctagcgcctatgatactgtttccggcatcgaaaacgtggtaggcactgaactggcagatcgtatcacgggtgacgcgcaagcgaacgttctgcgtggtgctggcggtgcagacgtgctggcgggtggtgaaggcgacgacgtactgctgggcggtgacggtgacgatcagctgagcggtgatgcgggtcgtgaccgtctgtacggtgaagcaggtgacgattggttcttccaggacgctgctaacgctggcaacctgctggacggcggcgatggccgtgacactgttgacttcagcggtccgggtcgcggtctggacgcaggcgcgaaaggcgttttcctgagcctgggtaagggtttcgcatctctgatggacgaaccagaaaccagcaatgtcctgcgtaacatcgaaaacgctgtcggttccgcacgcgatgatgttctgattggcgatgctggtgccaatgttctgaacggtctggcgggtaatgatgtactgtctggtggcgctggtgatgatgtcctgctgggtgatgagggcagcgatctgctgtccggcgatgccggcaacgacgatctgttcggtggccaaggtgatgatacttacctgttcggcgttggctatggtcatgacaccatctatgagtctggtggcggccacgatacgattcgtattaatgcaggtgctgaccaactgtggtttgcacgtcagggtaacgatctggaaattcgcatcctgggtactgatgacgctctgaccgtacacgactggtaccgcgatgcggatcaccgcgtagaaatcatccatgcggctaaccaagctgtagaccaggctggtattgaaaaactggtagaagcgatggcacagtatccggacccaggtgcggctgctgcagcacctccagcggctcgtgtgccggataccctgatgcagagcctggccgtcaattggcgttaa

SEQ ID NO: 10 - protein, 1613 aa, synthetic construct
  REGION 1..1613: note = Synthetic Construct
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 11 - DNA, 4512 bp, synthetic construct
  misc_feature 1..4512: note = Nucleotide sequence encoding polypeptide 1-183 + 387-1706 of SEQ ID NO:2
  source 1..4512: mol_type = other DNA
  CDS 1..4512
atgcagcagtcccatcaggccggttacgcaaacgcagccgatcgtgaatctggtatcccggcagcggttctggatggtattaaggcagttgccaaagagaagaacgctaccctgatgttccgtctggtcaacccacactctacttccctgatcgcggaaggtgtggcgaccaaaggcctgggtgtgcacgccaaaagcagcgactggggtctgcaggcaggttatattccggtgaacccgaatctgtctaaactgttcggtcgtgccccggaggtgatcgcacgtgcagacaacgatgtcaactcttctctggcgcatggtcacaccgcggttgatctgaccctgtccaaagaacgcctggattacctgcgtcaggcgggcctggtgacgggcatggccgacggtgttgtagcgagcaaccacgcgggttatgaacaatttgagtttcgcgttaaagagacctctgacggtcgttacgcggtgcagtaccgccgcaaaggtggcgatgactttgaagctgtcaaagtgatcggtaacgctgctggaattccgggcctgcgccgtccgtccctgggcgcagtcgaacgtcaggactccggctacgattccctggatggcgttggctcccgctccttctccctgggcgaggtttctgacatggctgcggtggaagcggcagagctggaaatgacccgtcaagtgctgcacgcgggcgcacgtcaggacgatgctgagccgggcgtgtccggtgcgtctgcacactggggtcaacgtgccctgcagggtgctcaagctgtcgcggcagcgcagcgtctggtacatgcgatcgcactgatgacccagttcggtcgcgcaggttctaccaacactcctcaggaagcagctagcctgtctgctgctgttttcggtctgggcgaagcgtcttctgcggttgcagaaaccgtgagcggtttcttccgtggttctagccgctgggcgggtggttttggtgtagcaggtggcgctatggccctgggtggcggtatcgcggctgctgtcggcgcaggtatgagcctgaccgacgatgctcctgcgggccagaaagccgcagctggcgctgaaatcgcgctgcagctgactggtggtaccgttgaactggctagctctatcgcgctggctctggcagcggcacgtggcgtgacttctggcctgcaagtcgccggtgcctctgcgggcgctgctgcgggcgctctggcagctgcactgtcccccatggaaatctacggtctggtacagcagtctcactacgcagaccagctggataaactggcgcaggagtcttctgcatacggttacgaaggcgacgcactgctggcgcagctgtatcgtgacaaaactgctgctgaaggtgctgtggcaggcgtttctgcggtactgtctaccgttggcgctgcggtttctattgcggctgccgcatccgttgtaggtgcgccggttgctgtagttacctctctgctgactggtgcactgaacggtatcctgcgtggtgttcagcagcctattatcgaaaaactggcgaatgattatgcccgtaaaatcgacgaactgggcggtccgcaggcttattttgaaaaaaacctgcaggcgcgccacgaacagctggcaaacagcgacggcctgcgcaaaatgctggcagacctgcaagctggttggaacgcgtcttctgtgatcggcgtgcagaccaccgaaattagcaaatctgcgctggaactggctgcaattactggcaacgcggataacctgaaaagcgttgatgtttttgtcgatcgcttcgttcagggcgagcgcgttgctggtcagccggttgttctggacgttgcggcaggcggcatcgacatcgctagccgcaagggcgagcgtccggctctgactttcattaccccgctggcagctccgggtgaagaacagcgtcgccgtaccaagactggtaaaagcgagttcaccaccttcgtggaaatcgtaggtaaacaggaccgctggcgtattcgcgacggtgcggcggacaccaccatcgacctggcaaaagttgtttcccaactggtagatgctaacggcgtgctgaaacattctattaaactggatgtaatcggcggtgacggcgatgacgttgtactggcgaacgcgtctcgtatccactatgacggcggcgcaggtaccaacacggtttcctacgcagcgctgggccgccaggattccatcactgttagcgccgatggcgaacgtttcaacgtgcgtaaacagctgaataacgcaaacgtctatcgtgagggtgtagctacccagaccaccgcgtacggtaagcgtaccgaaaacgttcagtatcgtcacgtggaactggcacgtgtgggtcagctggtagaggtggatacgctggaacacgtgcagcacatcatcggtggcgccggtaacgattccattactggtaacgctcacgacaacttcctggcaggtggttctggtgatgatcgtctggacggtggtgcgggtaacgatacgctggtaggcggcgaaggtcagaacaccgtaattggtggtgccggtgacgacgttttcctgcaggatctgggcgtttggagcaaccagctggatggcggtgccggtgttgacacggttaagtataacgttcaccagccgtctgaagagcgcctggaacgtatgggtgacactggtattcatgccgatctgcagaaaggcactgttgaaaaatggcctgcgctgaacctgttctctgtggaccatgttaagaacattgaaaacctgcatggctctcgcctgaacgaccgtatcgctggtgacgaccaggataacgaactgtggggtcatgacggcaatgataccattcgcggtcgtggcggcgacgatattctgcgtggcggtctgggcctggataccctgtatggtgaagatggtaacgatattttcctgcaagacgatgaaacggtatctgatgacatcgacggtggtgcaggcctggacaccgtagattacagcgctatgatccatccgggtcgtatcgtagctccgcacgagtacggcttcggtatcgaggcggacctgtctcgtgaatgggttcgtaaagcgtccgcgctgggtgtggactactacgataacgttcgtaacgtggaaaacgtgatcggtacctccatgaaagacgtgctgattggcgacgcacaggccaacactctgatgggccagggcggtgatgacacggttcgcggcggcgacggtgatgacctgctgttcggtggtgacggcaacgacatgctgtacggcgacgcgggcaacgacaccctgtacggcggtctgggtgacgacactctggaaggtggtgcaggtaacgactggttcggccaaacccaggcacgcgaacacgacgtgctgcgtggtggcgacggcgtagacaccgtggattactcccaaactggcgctcacgcgggtatcgcggccggtcgtatcggtctgggcattctggccgatctgggtgccggccgtgtcgacaaactgggtgaggctggctctagcgcctatgatactgtttccggcatcgaaaacgtggtaggcactgaactggcagatcgtatcacgggtgacgcgcaagcgaacgttctgcgtggtgctggcggtgcagacgtgctggcgggtggtgaaggcgacgacgtactgctgggcggtgacggtgacgatcagctgagcggtgatgcgggtcgtgaccgtctgtacggtgaagcaggtgacgattggttcttccaggacgctgctaacgctggcaacctgctggacggcggcgatggccgtgacactgttgacttcagcggtccgggtcgcggtctggacgcaggcgcgaaaggcgttttcctgagcctgggtaagggtttcgcatctctgatggacgaaccagaaaccagcaatgtcctgcgtaacatcgaaaacgctgtcggttccgcacgcgatgatgttctgattggcgatgctggtgccaatgttctgaacggtctggcgggtaatgatgtactgtctggtggcgctggtgatgatgtcctgctgggtgatgagggcagcgatctgctgtccggcgatgccggcaacgacgatctgttcggtggccaaggtgatgatacttacctgttcggcgttggctatggtcatgacaccatctatgagtctggtggcggccacgatacgattcgtattaatgcaggtgctgaccaactgtggtttgcacgtcagggtaacgatctggaaattcgcatcctgggtactgatgacgctctgaccgtacacgactggtaccgcgatgcggatcaccgcgtagaaatcatccatgcggctaaccaagctgtagaccaggctggtattgaaaaactggtagaagcgatggcacagtatccggacccaggtgcggctgctgcagcacctccagcggctcgtgtgccggataccctgatgcagagcctggccgtcaattggcgttaa

SEQ ID NO: 12 - protein, 1503 aa, synthetic construct
  REGION 1..1503: note = Synthetic Construct
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 13 - protein, 1613 aa, synthetic construct; identical to SEQ ID NO: 10
  REGION 1..1613: note = Polypeptide 1-227 + 321-1706 of SEQ ID NO: 4

SEQ ID NO: 14 - protein, 1503 aa, synthetic construct; identical to SEQ ID NO: 12
  REGION 1..1503: note = Polypeptide 1-183 + 387-1706 of SEQ ID NO:4

SEQ ID NO: 15 - protein, 1613 aa, synthetic construct
  REGION 1..1613: note = Polypeptide 1-227 + 321-1706 of SEQ ID NO:6
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASESQMLTRGQLKEYIGQQRGEGYVFYENRAYGVAGKSLFDDGLGAAPGVPGGRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAVGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 16 - protein, 1503 aa, synthetic construct
  REGION 1..1503: note = Polypeptide 1-183 + 387-1706 of SEQ ID NO:6
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAVGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMGYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGEGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 17 - protein, 1612 aa, synthetic construct
  REGION 1..1612: note = Polypeptide 1-227 + 321-1705 of SEQ ID NO:8
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASESQMLTRGQLKEYIGQQRGEGYVFYENRAYGVAGKSLFDDGLGAAPGVPGRRSKSSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIGAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 18 - protein, 1502 aa, synthetic construct
  REGION 1..1502: note = Polypeptide 1-183 + 387-1705 of SEQ ID NO:8
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIGAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSADVFVDRFIQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLEVIGGDGDDVVLANASRIHYDGGAGTNTVSYAALGRQDSITVSADGERFNVRKQLNNANVYREGVATQKTAYGKRTENVQYRHVELARVGQLVEVDTLEHVQHIIGGAGNDSITGNAHDNFLAGGAGDDRLDGGAGNDTLVGGEGHNTVVGGAGDDVFLQDLGVWSNQLDGGAGVDTVKYNVHQPSEERLERMGDTGIHADLQKGTVEKWPALNLFSVDHVKNIENLHGSSLNDSIAGDDRDNELWGDDGNDTIHGRGGDDILRGGLGLDTLYGEDGNDIFLQDDETVSDDIDGGAGLDTVDYSAMIHAGKIVAPHEYGFGIEADLSEGWVRKAARRGMDYYDSVRSVENVIGTSMKDVLIGDAQANTLMGQGGDDTVRGGDGDDLLFGGDGNDMLYGDAGNDTLYGGLGDDTLEGGAGNDWFGQTPAREHDVLRGGAGVDTVDYSQAGAHAGVATGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGNDTVDFSGPGRGLDAGAKGVFLSLGKGFASLMDEPETSNVLRHIENAVGSVRDDVLIGDAGANVLNGLAGNDVLSGGAGDDVLLGDEGSDLLSGDAGNDDLFGGQGDDTYLFGAGYGHDTIYESGGGHDTIRINAGADQLWFARQGNDLEIRILGTDDALTVHDWYRDADHRVEAIHAANQAIDPAGIEKLVEAMAQYPDPGAAAAAPPAARVPDTLMQSLAVNWR

SEQ ID NO: 19 - protein, 1547 aa, synthetic construct
  REGION 1..1547: note = Polypeptide 1-227 + 387-1706 of SEQ ID NO:2
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIPLTADIDMFAIMPHLSNFRDSARSSVTSGDSVTDYLARTRRAASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 20 - protein, 1569 aa, synthetic construct
  REGION 1..1569: note = polypeptide 1-183 + 321-1706 of SEQ ID NO:2
MQQSHQAGYANAADRESGIPAAVLDGIKAVAKEKNATLMFRLVNPHSTSLIAEGVATKGLGVHAKSSDWGLQAGYIPVNPNLSKLFGRAPEVIARADNDVNSSLAHGHTAVDLTLSKERLDYLRQAGLVTGMADGVVASNHAGYEQFEFRVKETSDGRYAVQYRRKGGDDFEAVKVIGNAAGIESQMLTRGQLKEYIGQQRGEGYVFYENRAYGVAGKSLFDDGLGAAPGVPSGRSKFSPDVLETVPASPGLRRPSLGAVERQDSGYDSLDGVGSRSFSLGEVSDMAAVEAAELEMTRQVLHAGARQDDAEPGVSGASAHWGQRALQGAQAVAAAQRLVHAIALMTQFGRAGSTNTPQEAASLSAAVFGLGEASSAVAETVSGFFRGSSRWAGGFGVAGGAMALGGGIAAAVGAGMSLTDDAPAGQKAAAGAEIALQLTGGTVELASSIALALAAARGVTSGLQVAGASAGAAAGALAAALSPMEIYGLVQQSHYADQLDKLAQESSAYGYEGDALLAQLYRDKTAAEGAVAGVSAVLSTVGAAVSIAAAASVVGAPVAVVTSLLTGALNGILRGVQQPIIEKLANDYARKIDELGGPQAYFEKNLQARHEQLANSDGLRKMLADLQAGWNASSVIGVQTTEISKSALELAAITGNADNLKSVDVFVDRFVQGERVAGQPVVLDVAAGGIDIASRKGERPALTFITPLAAPGEEQRRRTKTGKSEFTTFVEIVGKQDRWRIRDGAADTTIDLAKVVSQLVDANGVLKHSIKLDVIGGDGDDVVLANASRIHYDGGAGTNTVSYAALGRQDSITVSADGERFNVRKQLNNANVYREGVATQTTAYGKRTENVQYRHVELARVGQLVEVDTLEHVQHIIGGAGNDSITGNAHDNFLAGGSGDDRLDGGAGNDTLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDTIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDTLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTIYESGGGHDTIRINAGADQLWFARQGNDLEIRILGTDDALTVHDWYRDADHRVEIIHAANQAVDQAGIEKLVEAMAQYPDPGAAAAAPPAARVPDTLMQSLAVNWR

SEQ ID NO: 21 - DNA, 558 bp, Bordetella pertussis
  source 1..558: mol_type = unassigned DNA
atgcttccgtccgcccaagcgccctccctcctcaatcccaccgacgacttcgcggcactgggcaatattgcctggctgtggatgaactctcccatgcaccgcgactggccggtgcatctgctcgcacgcaacacgctcgcgccgattcaactgggccaatacattctgctgcgatgcaatgacgtgccggttgcatactgcagctgggccctaatggacgccgacaccgaactctcctatgtcatggcgccctcgtcgctgggcgggaatgcctggaactgcggcgaccgactgtggatcatcgactggatcgcgccattctcgcgcgacgacaatcgtgcgctgcgccgcgcgctggccgaacggcaccccgacagcgtgggccgttcgctgcgcgttcggcgcggcggcgacaccgcgcgcgtcaaggagtaccgaggccgcgcgctggacgcggccgccgctcgcgcgcagctggaccgctaccatgccgaactgatcgcaggactgcgcgcgagcaacggcggatacgcgccgcgaggccggggcaccgcctga

SEQ ID NO: 22 - DNA, 558 bp, synthetic construct
  misc_feature 1..558: note = Optimized version of CyaC nucleotide coding sequence
  source 1..558: mol_type = other DNA
  CDS 1..558
atgctgccgtctgcacaggcgccgtctctgctgaatccaactgatgacttcgctgcgctgggtaacatcgcgtggctgtggatgaacagcccgatgcaccgtgactggccggttcacctgctggcacgtaacacgctggcgccgatccagctgggtcaatacatcctgctgcgttgtaacgacgtgcctgtggcgtattgctcctgggcactgatggatgcggataccgagctgtcctacgttatggccccatctagcctgggcggtaacgcttggaactgcggcgatcgtctgtggatcattgattggattgctccgtttagccgtgacgacaaccgtgctctgcgtcgcgcgctggccgaacgtcatccggattctgttggccgttccctgcgcgtacgtcgcggtggcgacaccgctcgcgtcaaagaataccgtggtcgtgctctggacgcagcggcggcccgcgctcagctggaccgctaccacgcagaactgattgccggtctgcgtgcatccaatggcggttatgctccgcgtggccgtggcaccgcataa

SEQ ID NO: 23 - protein, 185 aa, synthetic construct
  REGION 1..185: note = Synthetic Construct
MLPSAQAPSLLNPTDDFAALGNIAWLWMNSPMHRDWPVHLLARNTLAPIQLGQYILLRCNDVPVAYCSWALMDADTELSYVMAPSSLGGNAWNCGDRLWIIDWIAPFSRDDNRALRRALAERHPDSVGRSLRVRRGGDTARVKEYRGRALDAAAARAQLDRYHAELIAGLRASNGGYAPRGRGTA

SEQ ID NO: 24 - DNA, 5121 bp, synthetic construct
  misc_feature 1..5121: note = Nucleotide sequence encoding the wild type CyaA protein of Bordetella pertussis, optimized for E. coli expression
  source 1..5121: mol_type = other DNA
atgcagcagtcccatcaggccggttacgcaaacgcagccgatcgtgaatctggtatcccggcagcggttctggatggtattaaggcagttgccaaagagaagaacgctaccctgatgttccgtctggtcaacccacactctacttccctgatcgcggaaggtgtggcgaccaaaggcctgggtgtgcacgccaaaagcagcgactggggtctgcaggcaggttatattccggtgaacccgaatctgtctaaactgttcggtcgtgccccggaggtgatcgcacgtgcagacaacgatgtcaactcttctctggcgcatggtcacaccgcggttgatctgaccctgtccaaagaacgcctggattacctgcgtcaggcgggcctggtgacgggcatggccgacggtgttgtagcgagcaaccacgcgggttatgaacaatttgagtttcgcgttaaagagacctctgacggtcgttacgcggtgcagtaccgccgcaaaggtggcgatgactttgaagctgtcaaagtgatcggtaacgctgctggaattccgctgaccgcggatatcgacatgtttgccatcatgcctcacctgtccaactttcgcgattccgcacgcagctctgtgacgtctggtgattccgtcactgactacctggctcgtacccgtcgtgcggcctctgaagccactggtggtctggatcgtgaacgcattgacctgctgtggaaaatcgcccgtgccggtgcgcgctctgctgttggtaccgaagcacgccgccaatttcgttacgacggcgatatgaacatcggtgtgatcacggattttgaactggaagttcgcaatgccctgaaccgtcgtgcacacgcggtcggcgcgcaggacgttgttcagcacggtacggagcagaataacccgttcccagaagcggacgaaaagatctttgttgtgtctgccaccggtgaatcccagatgctgactcgtggccagctgaaagaatacatcggccaacagcgtggcgaaggctacgttttctacgagaatcgtgcttacggcgttgcgggcaaatccctgttcgacgatggtctgggcgctgcaccgggcgttccgagcggccgttctaaattcagcccggacgtactggaaactgtgccggcttccccgggcctgcgccgtccgtccctgggcgcagtcgaacgtcaggactccggctacgattccctggatggcgttggctcccgctccttctccctgggcgaggtttctgacatggctgcggtggaagcggcagagctggaaatgacccgtcaagtgctgcacgcgggcgcacgtcaggacgatgctgagccgggcgtgtccggtgcgtctgcacactggggtcaacgtgccctgcagggtgctcaagctgtcgcggcagcgcagcgtctggtacatgcgatcgcactgatgacccagttcggtcgcgcaggttctaccaacactcctcaggaagcagctagcctgtctgctgctgttttcggtctgggcgaagcgtcttctgcggttgcagaaaccgtgagcggtttcttccgtggttctagccgctgggcgggtggttttggtgtagcaggtggcgctatggccctgggtggcggtatcgcggctgctgtcggcgcaggtatgagcctgaccgacgatgctcctgcgggccagaaagccgcagctggcgctgaaatcgcgctgcagctgactggtggtaccgttgaactggctagctctatcgcgctggctctggcagcggcacgtggcgtgacttctggcctgcaagtcgccggtgcctctgcgggcgctgctgcgggcgctctggcagctgcactgtcccccatggaaatctacggtctggtacagcagtctcactacgcagaccagctggataaactggcgcaggagtcttctgcatacggttacgaaggcgacgcactgctggcgcagctgtatcgtgacaaaactgctgctgaaggtgctgtggcaggcgtttctgcggtactgtctaccgttggcgctgcggtttctattgcggctgccgcatccgttgtaggtgcgccggttgctgtagttacctctctgctgactggtgcactgaacggtatcctgcgtggtgttcagcagcctattatcgaaaaactggcgaatgattatgcccgtaaaatcgacgaactgggcggtccgcaggcttattttgaaaaaaacctgcaggcgcgccacgaacagctggcaaacagcgacggcctgcgcaaaatgctggcagacctgcaagctggttggaacgcgtcttctgtgatcggcgtgcagaccaccgaaattagcaaatctgcgctggaactggctgcaattactggcaacgcggataacctgaaaagcgttgatgtttttgtcgatcgcttcgttcagggcgagcgcgttgctggtcagccggttgttctggacgttgcggcaggcggcatcgacatcgctagccgcaagggcgagcgtccggctctgactttcattaccccgctggcagctccgggtgaagaacagcgtcgccgtaccaagactggtaaaagcgagttcaccaccttcgtggaaatcgtaggtaaacaggaccgctggcgtattcgcgacggtgcggcggacaccaccatcgacctggcaaaagttgtttcccaactggtagatgctaacggcgtgctgaaacattctattaaactggatgtaatcggcggtgacggcgatgacgttgtactggcgaacgcgtctcgtatccactatgacggcggcgcaggtaccaacacggtttcctacgcagcgctgggccgccaggattccatcactgttagcgccgatggcgaacgtttcaacgtgcgtaaacagctgaataacgcaaacgtctatcgtgagggtgtagctacccagaccaccgcgtacggtaagcgtaccgaaaacgttcagtatcgtcacgtggaactggcacgtgtgggtcagctggtagaggtggatacgctggaacacgtgcagcacatcatcggtggcgccggtaacgattccattactggtaacgctcacgacaacttcctggcaggtggttctggtgatgatcgtctggacggtggtgcgggtaacgatacgctggtaggcggcgaaggtcagaacaccgtaattggtggtgccggtgacgacgttttcctgcaggatctgggcgtttggagcaaccagctggatggcggtgccggtgttgacacggttaagtataacgttcaccagccgtctgaagagcgcctggaacgtatgggtgacactggtattcatgccgatctgcagaaaggcactgttgaaaaatggcctgcgctgaacctgttctctgtggaccatgttaagaacattgaaaacctgcatggctctcgcctgaacgaccgtatcgctggtgacgaccaggataacgaactgtggggtcatgacggcaatgataccattcgcggtcgtggcggcgacgatattctgcgtggcggtctgggcctggataccctgtatggtgaagatggtaacgatattttcctgcaagacgatgaaacggtatctgatgacatcgacggtggtgcaggcctggacaccgtagattacagcgctatgatccatccgggtcgtatcgtagctccgcacgagtacggcttcggtatcgaggcggacctgtctcgtgaatgggttcgtaaagcgtccgcgctgggtgtggactactacgataacgttcgtaacgtggaaaacgtgatcggtacctccatgaaagacgtgctgattggcgacgcacaggccaacactctgatgggccagggcggtgatgacacggttcgcggcggcgacggtgatgacctgctgttcggtggtgacggcaacgacatgctgtacggcgacgcgggcaacgacaccctgtacggcggtctgggtgacgacactctggaaggtggtgcaggtaacgactggttcggccaaacccaggcacgcgaacacgacgtgctgcgtggtggcgacggcgtagacaccgtggattactcccaaactggcgctcacgcgggtatcgcggccggtcgtatcggtctgggcattctggccgatctgggtgccggccgtgtcgacaaactgggtgaggctggctctagcgcctatgatactgtttccggcatcgaaaacgtggtaggcactgaactggcagatcgtatcacgggtgacgcgcaagcgaacgttctgcgtggtgctggcggtgcagacgtgctggcgggtggtgaaggcgacgacgtactgctgggcggtgacggtgacgatcagctgagcggtgatgcgggtcgtgaccgtctgtacggtgaagcaggtgacgattggttcttccaggacgctgctaacgctggcaacctgctggacggcggcgatggccgtgacactgttgacttcagcggtccgggtcgcggtctggacgcaggcgcgaaaggcgttttcctgagcctgggtaagggtttcgcatctctgatggacgaaccagaaaccagcaatgtcctgcgtaacatcgaaaacgctgtcggttccgcacgcgatgatgttctgattggcgatgctggtgccaatgttctgaacggtctggcgggtaatgatgtactgtctggtggcgctggtgatgatgtcctgctgggtgatgagggcagcgatctgctgtccggcgatgccggcaacgacgatctgttcggtggccaaggtgatgatacttacctgttcggcgttggctatggtcatgacaccatctatgagtctggtggcggccacgatacgattcgtattaatgcaggtgctgaccaactgtggtttgcacgtcagggtaacgatctggaaattcgcatcctgggtactgatgacgctctgaccgtacacgactggtaccgcgatgcggatcaccgcgtagaaatcatccatgcggctaaccaagctgtagaccaggctggtattgaaaaactggtagaagcgatggcacagtatccggacccaggtgcggctgctgcagcacctccagcggctcgtgtgccggataccctgatgcagagcctggccgtcaattggcgttaa

SEQ ID NO: 25 - DNA, 11172 bp, synthetic construct
  misc_feature 1..11172: note = pGTP-gtCyaAd93-CyaCopt
  source 1..11172: mol_type = other DNA
atcgagatctgtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaattataattttgtttaactttaagaaggagatatacatatgcagcagtcccatcaggccggttacgcaaacgcagccgatcgtgaatctggtatcccggcagcggttctggatggtattaaggcagttgccaaagagaagaacgctaccctgatgttccgtctggtcaacccacactctacttccctgatcgcggaaggtgtggcgaccaaaggcctgggtgtgcacgccaaaagcagcgactggggtctgcaggcaggttatattccggtgaacccgaatctgtctaaactgttcggtcgtgccccggaggtgatcgcacgtgcagacaacgatgtcaactcttctctggcgcatggtcacaccgcggttgatctgaccctgtccaaagaacgcctggattacctgcgtcaggcgggcctggtgacgggcatggccgacggtgttgtagcgagcaaccacgcgggttatgaacaatttgagtttcgcgttaaagagacctctgacggtcgttacgcggtgcagtaccgccgcaaaggtggcgatgactttgaagctgtcaaagtgatcggtaacgctgctggaattccgctgaccgcggatatcgacatgtttgccatcatgcctcacctgtccaactttcgcgattccgcacgcagctctgtgacgtctggtgattccgtcactgactacctggctcgtacccgtcgtgcggcctctgaatcccagatgctgactcgtggccagcttaaggaatacatcggccaacagcgtggcgaaggctacgttttctacgagaatcgtgcttacggcgttgcgggcaaatccctgttcgacgatggtctgggcgctgcaccgggcgttccgagcggccgttctaaattcagcccggacgtactggaaactgtgccggcttccccgggcctgcgccgtccgtccctgggcgcagtcgaacgtcaggactccggctacgattccctggatggcgttggctcccgctccttctccctgggcgaggtttctgacatggctgcggtggaagcggcagagctggaaatgacccgtcaagtgctgcacgcgggcgcacgtcaggacgatgctgagccgggcgtgtccggtgcgtctgcacactggggtcaacgtgccctgcagggtgctcaagctgtcgcggcagcgcagcgtctggtacatgcgatcgcactgatgacccagttcggtcgcgcaggttctaccaacactcctcaggaagcagctagcctgtctgctgctgttttcggtctgggcgaagcgtcttctgcggttgcagaaaccgtgagcggtttcttccgtggttctagccgctgggcgggtggttttggtgtagcaggtggcgctatggccctgggtggcggtatcgcggctgctgtcggcgcaggtatgagcctgaccgacgatgctcctgcgggccagaaagccgcagctggcgctgaaatcgcgctgcagctgactggtggtaccgttgaactggctagctctatcgcgctggctctggcagcggcacgtggcgtgacttctggcctgcaagtcgccggtgcctctgcgggcgctgctgcgggcgctctggcagctgcactgtcccccatggaaatctacggtctggtacagcagtctcactacgcagaccagctggataaactggcgcaggagtcttctgcatacggttacgaaggcgacgcactgctggcgcagctgtatcgtgacaaaactgctgctgaaggtgctgtggcaggcgtttctgcggtactgtctaccgttggcgctgcggtttctattgcggctgccgcatccgttgtaggtgcgccggttgctgtagttacctctctgctgactggtgcactgaacggtatcctgcgtggtgttcagcagcctattatcgaaaaactggcgaatgattatgcccgtaaaatcgacgaactgggcggtccgcaggcttattttgaaaaaaacctgcaggcgcgccacgaacagctggcaaacagcgacggcctgcgcaaaatgctggcagacctgcaagctggttggaacgcgtcttctgtgatcggcgtgcagaccaccgaaattagcaaatctgcgctggaactggctgcaattactggcaacgcggataacctgaaaagcgttgatgtttttgtcgatcgcttcgttcagggcgagcgcgttgctggtcagccggttgttctggacgttgcggcaggcggcatcgacatcgctagccgcaagggcgagcgtccggctctgactttcattaccccgctggcagctccgggtgaagaacagcgtcgccgtaccaagactggtaaaagcgagttcaccaccttcgtggaaatcgtaggtaaacaggaccgctggcgtattcgcgacggtgcggcggacaccaccatcgacctggcaaaagttgtttcccaactggtagatgctaacggcgtgctgaaacattctattaaactggatgtaatcggcggtgacggcgatgacgttgtactggcgaacgcgtctcgtatccactatgacggcggcgcaggtaccaacacggtttcctacgcagcgctgggccgccaggattccatcactgttagcgccgatggcgaacgtttcaacgtgcgtaaacagctgaataacgcaaacgtctatcgtgagggtgtagctacccagaccaccgcgtacggtaagcgtaccgaaaacgttcagtatcgtcacgtggaactggcacgtgtgggtcagctggtagaggtggatacgctggaacacgtgcagcacatcatcggtggcgccggtaacgattccattactggtaacgctcacgacaacttcctggcaggtggttctggtgatgatcgtctggacggtggtgcgggtaacgatacgctggtaggcggcgaaggtcagaacaccgtaattggtggtgccggtgacgacgttttcctgcaggatctgggcgtttggagcaaccagctggatggcggtgccggtgttgacacggttaagtataacgttcaccagccgtctgaagagcgcctggaacgtatgggtgacactggtattcatgccgatctgcagaaaggcactgttgaaaaatggcctgcgctgaacctgttctctgtggaccatgttaagaacattgaaaacctgcatggctctcgcctgaacgaccgtatcgctggtgacgaccaggataacgaactgtggggtcatgacggcaatgataccattcgcggtcgtggcggcgacgatattctgcgtggcggtctgggcctggataccctgtatggtgaagatggtaacgatattttcctgcaagacgatgaaacggtatctgatgacatcgacggtggtgcaggcctggacaccgtagattacagcgctatgatccatccgggtcgtatcgtagctccgcacgagtacggcttcggtatcgaggcggacctgtctcgtgaatgggttcgtaaagcgtccgcgctgggtgtggactactacgataacgttcgtaacgtggaaaacgtgatcggtacctccatgaaagacgtgctgattggcgacgcacaggccaacactctgatgggccagggcggtgatgacacggttcgcggcggcgacggtgatgacctgctgttcggtggtgacggcaacgacatgctgtacggcgacgcgggcaacgacaccctgtacggcggtctgggtgacgacactctggaaggtggtgcaggtaacgactggttcggccaaacccaggcacgcgaacacgacgtgctgcgtggtggcgacggcgtagacaccgtggattactcccaaactggcgctcacgcgggtatcgcggccggtcgtatcggtctgggcattctggccgatctgggtgccggccgtgtcgacaaactgggtgaggctggctctagcgcctatgatactgtttccggcatcgaaaacgtggtaggcactgaactggcagatcgtatcacgggtgacgcgcaagcgaacgttctgcgtggtgctggcggtgcagacgtgctggcgggtggtgaaggcgacgacgtactgctgggcggtgacggtgacgatcagctgagcggtgatgcgggtcgtgaccgtctgtacggtgaagcaggtgacgattggttcttccaggacgctgctaacgctggcaacctgctggacggcggcgatggccgtgacactgttgacttcagcggtccgggtcgcggtctggacgcaggcgcgaaaggcgttttcctgagcctgggtaagggtttcgcatctctgatggacgaaccagaaaccagcaatgtcctgcgtaacatcgaaaacgctgtcggttccgcacgcgatgatgttctgattggcgatgctggtgccaatgttctgaacggtctggcgggtaatgatgtactgtctggtggcgctggtgatgatgtcctgctgggtgatgagggcagcgatctgctgtccggcgatgccggcaacgacgatctgttcggtggccaaggtgatgatacttacctgttcggcgttggctatggtcatgacaccatctatgagtctggtggcggccacgatacgattcgtattaatgcaggtgctgaccaactgtggtttgcacgtcagggtaacgatctggaaattcgcatcctgggtactgatgacgctctgaccgtacacgactggtaccgcgatgcggatcaccgcgtagaaatcatccatgcggctaaccaagctgtagaccaggctggtattgaaaaactggtagaagcgatggcacagtatccggacccaggtgcggctgctgcagcacctccagcggctcgtgtgccggataccctgatgcagagcctggccgtcaattggcgttaatggatccaggcctctttagaaggagatataccatgctgccgtctgcacaggcgccgtctctgctgaatccaactgatgacttcgctgcgctgggtaacatcgcgtggctgtggatgaacagcccgatgcaccgtgactggccggttcacctgctggcacgtaacacgctggcgccgatccagctgggtcaatacatcctgctgcgttgtaacgacgtgcctgtggcgtattgctcctgggcactgatggatgcggataccgagctgtcctacgttatggccccatctagcctgggcggtaacgcttggaactgcggcgatcgtctgtggatcattgattggattgctccgtttagccgtgacgacaaccgtgctctgcgtcgcgcgctggccgaacgtcatccggattctgttggccgttccctgcgcgtacgtcgcggtggcgacaccgctcgcgtcaaagaataccgtggtcgtgctctggacgcagcggcggcccgcgctcagctggaccgctaccacgcagaactgattgccggtctgcgtgcatccaatggcggttatgctccgcgtggccgtggcaccgcataatctagaattctttaattaaagatccggctgctaacaaagcccgaaaggaagctgagttggctgctgccaccgctgagcaatccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgactcatagctgtttcctgtgtgaaattgttatccgctcacaattccacacaacatacgagccggaagcataaagtgtaaagcctggggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgctagcgtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtcggaatttatgcggtgtgaaataccgcacagatgcgtaaggagaaaataccgcatcaggcgctcttccgcttcctcgctcactgactcgctgccaccccgtggcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagagg

(19) United States
(12) Patent Application Publication
(10) Pub. No.: US 2023/0165950 A1
(43) Pub. Date: Jun. 1, 2023

(54) IMMUNOGENIC COMPOSITION COMPRISING CYAA-DERIVED POLYPEPTIDE PROMOTING A TH1/TH17-ORIENTED IMMUNE RESPONSE

(71) Applicants: GENKYOTEX, SAINT-JULIEN-EN GENEVOIS (FR); SERUM INSTITUTE OF INDIA PRIVATE LIMITED, PUNE (IN)

(72) Inventors: Rémi PALMANTIER, GuIzerix (FR); Yolande MISSERI, Dremil-Lafage (FR); Caroline DIVEU-SADER, Paris (FR); Sunil GAIROLA, Hadapsar, Pune (IN); Manish GAUTAM, Panchkula (IN); Harish RAO, Hadapsar (IN); Umesh SHALIGRAM, Pune (IN)

(21) Appl. No.: 17/814,988

(22) Filed: Jul. 26, 2022

Related U.S. Application Data
(63) Continuation of application No. 16/468,624, filed on Jun. 11, 2019, now abandoned.

(30) Foreign Application Priority Data
Dec. 27, 2016 (EP) 16306822.4

(51) Int. Cl. A61K 39/02; A61K 9/00; A61K 39/05; A61K 39/08; A61K 39/102; A61K 39/13; A61K 39/29; A61K 39/39; A61K 39/00

(52) U.S. Cl. CPC A61K 39/099; A61K 9/0019; A61K 39/05; A61K 39/08; A61K 39/102; A61K 39/13; A61K 39/292; A61K 39/39; C12Y 406/01001; A61K 39/0018; A61K 2039/55505; A61K 2039/55561; A61K 2039/55572; A61K 2039/55594; C12N 2730/10134; C12N 2770/32634; A61K 2039/10

(57) ABSTRACT

The invention relates to the use of a polypeptide derived from the adenylate cyclase of a Bordetella sp. (CyaA-derived polypeptide) by deletion of a segment of at least 93 amino acid residues, in particular a polypeptide derived from CyaA of Bordetella pertussis, as an immunomodifying antigen of the TH1/TH17-oriented immune response in an immunogenic composition. The invention relates to a vaccine candidate comprising such CyaA-derived polypeptide, either in an acellular immunogenic composition for active immunization against a condition causally related to the infection of a host by Bordetella sp. or in a combination composition encompassing said acellular immunogenic composition.

The sequence listing in the Sequence Listing XML file named EGP004 created on Jan. 22, 2023, which is 97.8 KB, is incorporated herein by reference.

The invention relates to the use of a polypeptide derived from the adenylate cyclase of a Bordetella sp. (CyaA-derived polypeptide), in particular of Bordetella pertussis, as an agent promoting the TH1/TH17-oriented immune response in an immunogenic composition. In particular, the invention relates to an immunogenic composition comprising said CyaA-derived polypeptide for the preparation of an acellular candidate vaccine against a condition causally related to the infection by a Bordetella sp. or for the preparation of a combination candidate vaccine encompassing the latter in association with other active ingredients for active immunization.

Whooping cough (pertussis) is a vaccine-preventable disease caused by the bacterium Bordetella pertussis. The first pertussis vaccines were developed during the 30's and implemented in the developed countries during the 40's and 50's (1). These were whole cell vaccines (WCV or wP) composed of chemically killed bacteria and combined with diphtheria and tetanus toxoids (DTwP) in the late 40's. Between 1950 and 1960, large-scale vaccination with the wP vaccine was followed by a dramatic reduction (>90%) of incidence and mortality of pertussis in industrialized world. However, wP vaccines were reactogenic causing seizures, neurological diseases and other side effects (2).

To avoid those side effects, a second generation of pertussis vaccines, acellular pertussis vaccines (aP) was developed in the early 1980s. The aP vaccines included from 1 to 5 purified antigens from Bordetella pertussis, e.g. pertussis toxin (PT) and adhesins such as filamentous hemagglutinine (FHA), pertactin (PRN) and fimbrial agglutinogens (AG). aP vaccines have been used in Japan since 1981 (3) and have been recommended for boosters in the United States since 1991 (4). Because of their safety profile, many developed countries switched to the use of aP for the entire vaccination course at the end of the 1990's and the early 2000's (5).

Since the introduction of aP vaccines, the scientific community has observed a resurgence of whooping cough cases (5-8). In 2008, estimates from WHO suggest that about 16 million cases of pertussis occurred worldwide, and that about 195 000 children died from this disease (9).

aP vaccines have been described to be protective against clinical symptoms of pertussis but not against colonization and transmission of the pathogen in contrast with WCV (14). This observation is supported by a distinct immune response induced by both vaccine types. aP vaccination induces a TH2-oriented immune response whereas WCV induces a TH1 and Th17-oriented immune response (15). This was recently supported by an experiment in a baboon model, where a correlation was made between the absence of a good TH1 response in aP vaccination, the shorter duration of protection and the maintenance of the bacteria since it can colonize lungs of vaccinated animals without inducing the disease (14).

Several studies indicate that aP-induced immunity is shorter in duration than wP- or infection induced immunity, resulting in an increased susceptibility to the infection for those vaccinated with aP compared to those vaccinated with WCV (10, 11). Children that had DTaP as primary vaccination and subsequent doses are more prone to disease that those who received DTwP vaccine as illustrated by a study in the US where after the fifth dose of DTaP, the odds of acquiring pertussis increased by an average of 42% per year (12). This rapid waning immunity has also been described when using aP as boosters (13). Based on a large population of persons aged 8-20 years with a high attack rate due to pertussis outbreaks in the early 2010s in the US and UK, Witt et al showed that there was an 8.57 relative risk (RR) of pertussis (P<0.0001) for patients who received only aP vaccines in contrast to those with at least one dose of wP during their pertussis vaccination history (14). In addition, the administration of 1 or more wP doses markedly augmented significantly the durability of immunity from subsequent aP doses.

The difference in the protection and duration may indeed result from the different types of immune responses that are induced after wP and aP. For instance, aP and wP vaccination induce functionally different T cell responses to pertussis. Noteworthy, the Th1 or Th2 bias induced by wP or aP primary vaccination, respectively, become fixed and are unchanged upon boosting with Tdap (Bancroft et al, 2016) or even further enhanced in the case of the aP-induced Th2 response (Ryan et al, 2000). Similar observations have been made in mice. These works have evaluated different types of adjuvants (Allen et Mills, Expert Review of Vaccines, 13(10)2014) showing that protection to B. pertussis challenge was improved in the presence of Th1-oriented adjuvants. In these challenges the injection routes of the vaccines are in majority intra-peritoneal sometimes sub-cutaneous or intra-nasal. The same authors presented at the 11th International Bordetella symposium (Apr. 5-8, 2016 Buenos Aires, Argentina; http://bordetella2016.com.ar/en/images/Programa-Bordetella-Symposium.pdf) results with a prime boost setting where the protection against a B. pertussis challenge of animals immunized with aP+LP1569 (LP1569 is a TLR2 agonist used as adjuvant prone to induce Th1 or Th17 or both immune responses (TH1/TH17)) and boosted with the same product was obtained in trachea and lungs whereas a prime with aP+alum and a boost with aP+LP1569 was not able to confer this protection. The authors concluded that despite the presence of the Th1-type adjuvant in the boost vaccine the immune response and consequent protection was equivalent to the group vaccinated with aP+alum and boosted with aP+alum.

From these observations, it is understood that the type of immune response induced against the antigens of an acellular Pertussis vaccine is one critical factor for the clinical efficacy of the protection of the host, in particular for a sustained protection over time. The identification of agents that may influence the orientation of the immune response toward a TH1 response has been looked for several years and interest has been accordingly directed to properties of one of Pertussis proteins, i.e., adenylate cyclase.

The Adenylate Cyclase of Bordetella pertussis (CyaA) is a toxin-hemolysin, a protein responsible for immune neutralization of the host's immune system since it targets professional immune cells by binding with high affinity to the CD11b/CD18 integrin receptor which is expressed on innate immune cells, including Antigen-presenting cells (APCs) as macrophages and dendritic cells (DC) (16). Within cells, CyaA is able to suppress host antibactericidal activity thereby promoting bacterial colonization and persistence (17-20). Accordingly detoxified or non-toxic CyaA was preferred in order to assess its properties in relation to immune response.

Different works with detoxified or native CyaA have shown several properties of the molecule. Conclusions and administration protocols of published experiments are disclosed hereafter.
MacDonald-Fyall et al. (21) describe the adjuvant activity of the detoxified CyaA but this adjuvant activity seems to be lost against Pertussis Toxin (PT) when CyaA is added to the vaccine;
Cheung et al. (22) describe no significant variation of the total level of IgG antibody responses to PT, FHA and PRN in mice immunized at day 0 and day 28 with an aP vaccine+different forms of CyaA (proCyaA, detoxified CyaA and native CyaA). They describe significant greater levels of IgG2a (TH1 response) to PRN in those mice vaccinated with acellular pertussis vaccine+detoxified CyaA. They also show an increase in aP-specific IFNg response following 2 immunizations with DTaP ⅛ containing ⅛ of the human dose of Aluminium (i.e. 0.0625 mg Al⁺⁺⁺/injection) in the presence of detoxified CyaA compared to the same DTaP in the absence of CyaA.
Dunne et al., (23) describe the capacity of CyaA to promote an IL-1β-mediated Th17 immune response protective in a respiratory challenge model with WT B. pertussis in mice. They show that CyaA co-injected with KLH, in mice with no addition of conventional vaccine adjuvant such as Aluminum salts induces a specific KLH-TH17 response.
Dadaglio et al. (24), describe CyaA as an important Toll like receptor (TLR) 4-signaling factor in the induction of protective responses against B. pertussis through its induction of Th1 T-cells and cytotoxic T lymphocytes (CTL) responses independently of lipopolysaccharide (LPS);

| | Administration | | Vaccination | | |
|---|---|---|---|---|---|
| Mouse immunization with | route | Adjuvant | schedule | Main results | Reference |
| 1-PT (120 ng), FHA (25 ng), | Intra- | No adj. | Prime | Induction of CyaA and | MacDonald- |
| PRN (10 μg) (per dose) +/− | peritoneal | | | CyaA* neutralizing Ab. | Fyall et al., |
| CyaA or CyaA* (15 | | | | Co-administration with | 2004 |
| μg/dose) [1] | | | | CyaA*: increase of Ab | |
| 2-PTd [2], FHA, PRN each at | | | | response against PT, FHA | |
| 10 μg/dose +/− CyaA or | | | | and PRN but inclusion of PTd | |
| CyaA* (15 μg/dose) | | | | with CyaA* reduces adjuvant | |
| | | | | effect of CyaA* on Ab | |
| | | | | response to other Ag. | |
| | | | | CyaA*+ Ag activates | |
| | | | | macrophages | |
| | | | | (NO production) | |
| | | | | CyaA* and CyaA when | |
| | | | | mixed with B. pertussis Ag | |
| | | | | increase IFNg production | |
| 1-CyaA + Al(OH)₃ | Intra- | Aluminum | Day 0 | No significant influence of | Cheung et al., |
| 2-CyaA + 1/8 DTap [3] | peritoneal | hydroxide | and | total IgG antibody response | 2006 |
| Infanrix ™, GSK) | | (Al(OH)₃) | day 28 | to PT, FHA and PRN | |
| 3-DTap | | | | Dtap + CyaA*: increased | |
| | | | | IgG2a to PRN | |
| | | | | CyaA* + Dtap: CyaA* appeared | |
| | | | | to promote a mixed Th1/Th2 | |
| | | | | response to B. pertussis | |
| | | | | antigens (compared to CyaA + | |
| | | | | Dtap and different pro-CyaA | |
| | | | | forms): increased production of | |
| | | | | IL5, Il6, GM-CSF and IFN-g | |
| | | | | from spleen cells and NO from | |
| | | | | macrophages. | |
| 1-KLH (5 μg/mouse) +/−CyaA | Sub- | PBS | Day 0 | CyaA induces production of | Dunne et al., |
| (1 μg/mouse) | cutaneous | | | IL-1beta. | 2010 |
| 2-CyaA alone | | | | CyaA activates the inflammasome | |
| | | | | independently of CyaA cytotoxic | |
| | | | | activity. | |
| | | | | CyaA + KLH: CyaA drives | |
| | | | | KLH-specific IL-17 | |
| | | | | production in vivo. | |
| | | | | CyaA promotes the induction of | |
| | | | | B. pertussis-specific IL-17 | |
| | | | | production. | |
| 1-PT and FHA + Alum | Intra- | Alum or | Day 0 | Immunization with PT/FHA + | Dunne et al., |
| 2-PT and FHA + LP1569 | peritoneal | LP1569 | and | Alum didn't confer the | 2015 |
| 3-PT + FHA + PRN +/− | | | week 4 | appropriate lung protection | |
| LP1569 | | | Day 0 | compared to an immunization | |
| | | | and | with PT/FHA + LP1569 | |
| | | | week 5 | PT/FHA/PRN + Alum induce a | |
| | | | | TH2 immune response compared | |
| | | | | to PT/FHA + LP1569 (Figure 7) | |
| 1-CyaA-Tyr | Intravenous | No | D0 | highly purified CyaA-Tyr is able | Dadaglio et al, |
| 2-CyaA-OVA | | adjuvant | | to generate efficient CD8+ T cell | 2014 |
| 3-CyaA-A488 | | | | responses in vivo in the absence | |
| | | | | of any adjuvant | |
| | | | | the efficiency of CyaA-Tyr in | |
| | | | | priming CD8+ T cells is due to | |
| | | | | its ability to activate cDC | |
| | | | | CyaA triggers DC activation | |
| | | | | through the TLR4/TRIF pathway | |
| | | | | the activation of CD8+ T cells by | |
| | | | | CyaA is independent of both the | |
| | | | | inflammasome and IL-1b | |

[1] Two Adenylate cyclases were tested, an enzymatically active form named CyaA and an enzymatically inactivated form named CyaA*.
[2] PTd: detoxified pertussis toxin
[3] one eigth of a single human dose of commercially available diphtheria, tetanus and acellular pertussis vaccine from GSK (25 μg dPT, 25 μg FHA, 8 μg PRN, 30 IU of diphtheria toxoid and 40 IU of tetanus toxoid with 0.5 mg of Al(OH)₃ per single human dose.

The experiments reported in the literature, involving the use of CyaA or detoxified CyaA in immunogenic compositions provide contrasted results regarding the influence of CyaA or detoxified CyaA proteins and do not allow to direct further searches in a clear direction as many parameters likely influence the elicitation of an immune response of a type favorable to a strong and lasting protection. Needs for design of new vaccine candidates and suitable administration settings in order to achieve protection thus remain.

### SUMMARY OF THE INVENTION

Beyond selection of a CyaA polypeptide that might be effective in eliciting or in promoting a response to the antigens of a Pertussis vaccine in the context of its administration to a host, the inventors have observed that results achieved in the prior art (such as results disclosed in Cheung et al) could not be reproduced in conditions where the TH2 adjuvant (alum) was in concentration similar to its concentration in doses of human vaccine and also in conditions wherein the animals are primed with TH2 polarizing Acellular Pertussis based vaccines. The inventors have however elucidated appropriate conditions for assessment of the immune response of administered acellular Pertussis vaccines, including in the context of combination vaccines encompassing aP and shown that non-toxic CyaA may be a successful immunomodifier for the design of vaccination settings suitable for administration routes used in human hosts. From their experimental results, the inventors have for the first time shown that a non-toxic CyaA, such as the GTL003 used herein provides better anti-CyaA-specific antibody titres, IFN gamma and IL-17 responses when comprised in a vaccine candidate with respect to whole cell vaccine as well as acellular pertussis vaccine that does not contain such non-toxic CyaA polypeptide. Accordingly using a non-toxic CyaA polypeptide will favorably influence protective responses against infection by Bordetella strains in the mucosa and the lungs, including for immune response required for a sustained protection.

The inventors have accordingly determined the influence of a non-toxic CyaA, in particular of a CyaA-derived polypeptide which is non-toxic as a result of a deletion of an amino acid segment of the sequence of the native CyaA protein and have assessed such non-toxic CyaA-derived polypeptide in conditions that appear close to administration conditions of available human aP vaccines. Based on results obtained they have been able to devise new compositions comprising detoxified CyaA, in particular recombinant genetically detoxified CyaA-derived polypeptides as described in WO 2014/016310 (and named hereafter GTL003) which have been associated with acellular vaccine antigens of Bordetella pertussis in the context of a combination Tdap vaccine adjuvanted with an adjuvant prone to induce primarily TH2 immune responses such as aluminum hydroxide at the human dose of the Tdap vaccine (0.39 mg Al⁺⁺⁺/injection). Unexpectedly, despite the presence of this TH2 adjuvant, the addition of GTL003 to the Tdap vaccine allowed to obtain an antigen-specific cytokine response bias toward TH1/TH17 profile following injection(s) after the first injection administered in mice.

The inventors have accordingly shown that GTL003, as an illustrative example of non-toxic CyaA, when present in an immunogenic composition containing a TH2 type aluminum adjuvant has the previously unknown capacity to induce a cytokine profile of the TH1/TH17 type, favorable for the generation of effector and memory T-lymphocytes resulting in TH1 IgG isotypes production and the induction of a more protective mucosal immunity, which are key factors in anti-bacterial infection. This crucial orientation of the immune response and possible additional effect on the level of antibodies raised against the antigens of Pertussis is an unexpected result that enables to develop a new generation of aP vaccines including a Tdap vaccine that may especially be used for the second and further immunizations when a CyaA-derived polypeptide such as GTL003 promotes a response suitable for higher and long-lasting protection.

The invention is directed to the use of a non-toxic CyaA, in particular a CyaA-derived polypeptide, for the manufacture of an immunogenic composition comprising active ingredients of the immune response of a combination vaccine, wherein said non-toxic CyaA, in particular CyaA-derived polypeptide is provided as an active ingredient of the protective immune response against diseases or conditions causally related to infection by Bordetella, in particular Bordetella pertussis, and/or is provided as an immunomodulatory agent or immunomodifier triggering the immune response for preventive protection against at least one of the active ingredients of the combination vaccine (preferably against all the active ingredients of the combination vaccine) toward a TH1- and/or a TH17-oriented immune response.

Such a non-toxic CyaA is a protein or a polypeptide that has lost its enzymatic adenylate cyclase activity. Non-toxic CyaA have been disclosed in the art, and may accordingly result from chemical or genetic modifications of the native protein of Bordetella.

In particular, it is a CyaA-derived polypeptide which is non cytotoxic polypeptide and is derived from the adenylate cyclase protein (CyaA-derived polypeptide) of a Bordetella strain, said polypeptide being (i) a polypeptide the amino acid sequence of which is obtained from CyaA of Bordetella pertussis and contains a deletion of a continuous segment of at least 93 amino acid residues from position 227 to position 321 in the amino acid sequence of the native CyaA of Bordetella pertussis or (ii) a polypeptide which is a variant of the polypeptide in (i) the amino acid sequence of said variant polypeptide being obtained from CyaA of Bordetella parapertussis, Bordetella bronchiseptica or Bordetella hinzii, and containing the deletion of a continuous segment of at least 93 amino acid residues defined by the positions in the native CyaA sequence of Bordetella parapertussis, Bordetella bronchiseptica or Bordetella hinzii matching those in CyaA of said continuous segment of at least 93 amino acid residues of Bordetella pertussis.

In a particular embodiment of the invention the immunogenic composition is chosen in the group of:
a) an acellular immunogenic composition suitable for the protection against a condition causally related to the infection by a Bordetella strain, in particular Bordetella pertussis in a human host, or
b) a combination immunogenic composition comprising said acellular immunogenic composition suitable for the protection against a condition causally related to the infection by a Bordetella strain, in particular Bordetella pertussis in a human host
wherein said acellular immunogenic composition comprises:
   a non-toxic polypeptide, in particular a non-cytotoxic polypeptide, derived from the adenylate cyclase protein (CyaA-derived polypeptide) of a Bordetella strain, said polypeptide being in a particular embodiment (i) a polypeptide the amino acid sequence of which is obtained from CyaA of Bordetella pertussis and contains a deletion of a continuous segment of at least 93 amino acid residues from position 227 to position 321 in the amino acid sequence of the native CyaA of Bordetella pertussis or (ii) a polypeptide which is a variant of the polypeptide in (i) in particular a variant the amino acid sequence of which is obtained from CyaA of Bordetella parapertussis, Bordetella bronchiseptica or Bordetella hinzii, and containing the deletion of a continuous segment of at least 93 amino acid residues defined by the positions in the native CyaA sequence of Bordetella parapertussis, Bordetella bronchiseptica or Bordetella hinzii matching positions 227 and 321 in CyaA of said continuous segment of at least 93 amino acid residues of Bordetella pertussis and,
   optionally an adjuvant of the TH1 immune response or an adjuvant of the TH2 immune response or a combination of both

In a particular aspect of the invention, the immunogenic composition is an acellular immunogenic composition suitable for the protection against a condition causally related to the infection by a Bordetella strain, in particular Bordetella pertussis, in a human, which comprises:
at least two antigens from a Bordetella strain wherein one antigen is (i) a non-toxic polypeptide, in particular a non-cytotoxic polypeptide, derived from the adenylate cyclase protein (CyaA-derived polypeptide) of a Bordetella strain said polypeptide being (i.1) either a polypeptide the amino acid sequence of which is obtained from CyaA of Bordetella pertussis and contains a deletion of a continuous segment of at least 93 amino acid residues from position 227 to position 321 in the amino acid sequence of the native CyaA of Bordetella pertussis, (i.2) or a polypeptide which is a variant of the polypeptide in (i.1) the amino acid sequence of said variant polypeptide being obtained from CyaA of Bordetella parapertussis, Bordetella bronchiseptica or Bordetella hinzii, and containing the deletion of a continuous segment of at least 93 amino acid residues defined by the positions in the native CyaA sequence of Bordetella parapertussis, Bordetella bronchiseptica or Bordetella hinzii matching positions 227 and 321 in CyaA of said continuous segment of at least 93 amino acid residues of Bordetella pertussis, and wherein further antigen(s) is (are) selected in the group of (ii) Pertussis toxoid (PT), (iii) Filamentous Haemagglutinin (FHA), (iv) Pertactin (PRN) and (v) Fimbriae (Fim),
wherein a protective immune response against the Bordetella strain is raised against said antigens and,
optionally an adjuvant of the TH1 immune response or an adjuvant of the TH2 immune response or a combination of both.

In another particular embodiment of the invention a combination immunogenic composition as herein defined further comprises antigens as active ingredients for the elicitation of an immune protection against determined pathogens which are (vi) at least one antigen of Clostridium tetani consisting of the Tetanus toxoid, (vii) an antigen of Corynebacterium complex consisting of the diphtheria toxoid, and optionally (viii) further antigens of different pathogen(s).

Additional antigens may further be included in the immunogenic composition to especially increase the valence of the immune response and accordingly a combination immunogenic composition may further comprise antigens as active ingredients for the elicitation of an immune protection against determined pathogens which are selected in the group of Hepatitis B surface antigen (HBs), inactivated poliovirus (IPV) of one or several virus strains, Haemophilus influenza type b polysaccharide.

Combination antigens originating from different pathogens are conventionally included in vaccine compositions administered to human hosts and should benefit from the particular properties of the CyaA-derived polypeptide as described herein to trigger the response toward a TH1 and/or TH17-oriented response even in the presence of the TH2 conventional adjuvants.

In a particular embodiment the immunogenic composition the antigens of Bordetella sp. are selected in the group of Pertussis toxoid (PT), Filamentous Haemagglutinin (FHA), Pertactin (PRN) and Fimbriae (Fim) are independently of each other from Bordetella pertussis, Bordetella parapertussis, Bordetella bronchiseptica or Bordetella hinzii, and are preferably from B. pertussis, and preferably are all from the same Bordetella strain.

A particular combination immunogenic composition according to the invention is such that one dose of 0.5 ml of the immunogenic composition contains:
Bordetella toxin in detoxified form (in particular either genetically or chemically detoxified), in particular Pertussis toxoid: 1 to 50 micrograms;
Filamentous Haemagglutinin: 1 to 50 μg;
Pertactin: 1 to 20 μg;
Optionally, Fimbriae: 2 to 25 rig;
Tetanus toxoid: at least 0.25 and less than 25 Lf;
Diphteria toxoid: at least 0.25 and less than 50 Lf;
   and optionally,
Hepatitis B surface antigen: 5 μg to 25 μg
inactivated poliovirus: from 5 to 45 D-antigen unit, in particular from 8 to 40 D-antigen unit per strain;
Haemophilus influenza type b polysaccharide: 1 to 20 μg

Advantageously, a particular combination composition is such that one dose of 0.5 ml of the immunogenic composition comprises:
Bordetella toxin as a detoxified form, in particular Bordetella toxoid in particular Pertussis toxoid: 8 μg;
Filamentous Haemagglutinin: 8 μg;
Pertactin: 2.5 μg;
Tetanus toxoid: 5 Lf;
Diphteria toxoid: 2.5 Lf.

In a specific embodiment the immunogenic composition, in particular the combination immunogenic composition, is further characterized in that one dose of 0.5 ml of the immunogenic composition comprises 2.5 to 600 μg of the CyaA-derived polypeptide, in particular 2.5 to 500 μg, 2.5 to 400 μg, 2.5 to 300 μg, 2.5 to 200 μg or 2.5 to 100 μg and preferably comprises 25 μg of the CyaA-derived polypeptide of Bordetella pertussis.

Having observed the immunomodifier function of the CyaA-derived polypeptide in accordance with the invention, the quantitative composition of the immunogenic composition may be adapted in particular for use of the composition such as when using it as a further dose in a multiple-dose setting or as a booster composition for a prime/boost vaccination schedule.

The presence of the CyaA-derived polypeptide in the immunogenic composition function has been shown both to elicit antibodies against said CyaA-derived polypeptide and to trigger orientation of the immune response toward TH1/TH17 response, in particular encompassing production of IL-17 when the composition is an acellular immunogenic composition consisting of active ingredients which comprise antigens of Bordetella sp. This should be in favor of the protection against whooping cough but also against various conditions causally related to the infection of the host by a Bordetella sp., in particular by Bordetella pertussis, such as protection against the transmission of the bacteria by the host to another individual or protection against the colonization of the airways of the infected host by the bacteria.

The immunogenic composition according to the invention may comprise adjuvant of several categories, including adjuvants of the TH2 response. Such adjuvants may be selected in the following groups:
An adjuvant of the TH2 response which is an aluminum salt, in particular an aluminum hydroxide, aluminum hydroxyphosphate sulfate or aluminum phosphate,
Adjuvant of TH1 response selected in the group of MPL-containing adjuvant, in particular AS15, AS01B, AS01D, or AS01E (provided by GSK); CpG-containing adjuvant, in particular AS15, QS21-containing adjuvant (provided by GSK), in particular AS01B, D, and E or AS15, immune stimulating complexes (ISCOMs), IC31-containing adjuvant, chitosan-containing adjuvant, liposomal formulation-based adjuvant, in particular AS01B D, E or AS15 or ISCOMs, lipid-based emulsions such as MF59,
An adjuvant inducing a TH1 response which is or contains a ligand of a toll-like receptor selected among:
   a ligand of toll-like receptor 4 (TLR-4), in particular monophosphoryl lipid A (MPL) or Glucopyranosyl Lipid A (GLA) or,
   a ligand of toll-like receptor 9 (TLR-9), in particular a synthetic oligodeoxynucleotides (ODNs) containing unmethylated CpG motifs and more particularly HBsAg-1018 or,
   a ligand of toll-like receptor 3 (TLR-3), in particular a synthetic analog of double-stranded RNA (dsRNA) and more particularly a composition containing Poly(I:C) such as polyinosinic-polycytidylic acid plus poly-L-lysine double-stranded RNA in the presence of carboxymethylcellulose (Poly (ICLC). or,
   a ligand of toll-like receptor 2 (TLR-2), in particular synthetic lipopeptides or a recombinant lipoprotein and,
   a ligand of toll-like receptor 5 (TLR-5), in particular a recombinant bacterial flagellin.

In an aspect of the immunogenic composition of the invention, the CyaA-derived polypeptide of Bordetella consists of:
a segment or a fragment of Bordetella pertussis CyaA protein as set forth in SEQ ID No. 2, the sequence of said segment or fragment beginning with the first residue of SEQ ID No.2 and ending with a residue located from position 183 to position 227 of SEQ ID No.2 or a polypeptide variant obtained from Bordetella parapertussis, Bordetella bronchiseptica or Bordetella hinzii and having at least 95% identity in amino acid residues with the segment or fragment consisting of residues 1 to 183 or 1 to 227 in SEQ ID No.2, fused to
a segment or a fragment of Bordetella pertussis CyaA protein as set forth in SEQ ID No. 2, the sequence of said fragment beginning with a residue located from position 321 to position 387 of SEQ ID No.2 and ending with the last residue of SEQ ID No.2 or a polypeptide variant obtained from Bordetella parapertussis, Bordetella bronchiseptica or Bordetella hinzii, and having at least 95% identity in amino acid residues with the segment or fragment consisting of residues 321 to final residue or 387 to final residue in SEQ ID No.2.

The invention also concerns a medicinal composition for administration to a human host which comprises an immunogenic composition according to any one of the embodiments disclosed herein and which is provided as an administration form selected among a powder, a powder and solution for reconstitution, a liquid, in particular a suspension or a solution, a lyophilized component and a combination of such administration forms.

In a particular embodiment, one dose of the medicinal composition for administration to a human host contains 0.25 ml to 1 ml of liquid, in particular of reconstituted product.

Various administration routes are available for administration to the host, including injection, preferably intramuscular injection.

The invention also concerns a method for preparing an immunogenic composition (also designated vaccine for the purpose of this disclosure) comprising as active ingredients for protection against determined pathogens or condition causally related to such pathogens, antigens of said pathogens and furthermore a polypeptide derived from the adenylate cyclase protein (CyaA-derived polypeptide) of a Bordetella strain as defined herein, wherein the method comprises the step(s) of:
a) providing antigens selected in the group of (i) at least one of the antigens from Bordetella selected in the group of Pertussis toxin in detoxified form (either genetically or chemically) (PT), Filamentous Haemagglutinin (FHA), Pertactin (PRN) and Fimbriae (Fim) and preferably the first three antigens or all of these antigens, (ii) at least one antigen of Clostridium tetani consisting of the Tetanus toxin in detoxified form (either genetically or chemically), (iii) an antigen of Corynebacterium bacteria complex consisting of the diphtheria toxin in detoxified form (either genetically or chemically), and (iv) the CyaA-derived polypeptide as defined in any one of the embodiments disclosed herein and optionally (v) further antigens of different pathogen(s) as active ingredients for the elicitation of an immune response against said determined pathogens formulated as one or several component(s) and,
b) admixing said component(s) with one or more compounds that enhance the immune response when the vaccine is administered to a host such as adjuvant(s) of the TH2-oriented immune response, adjuvant of the TH1-oriented immune response, or a combination of such adjuvants,

wherein steps a) and b) are optionally carried out as a single step.

Tetanus toxin in detoxified form encompasses Tetanus toxoid and Diphtheria toxin in detoxified form encompasses Diphtheria toxoid. These detoxified forms of toxins are known in the art, in particular from commercial vaccines against Tetanus and Diphtheria respectively.

According to an embodiment of the invention, an immunogenic composition as described in the present application is for use in active immunization of a human host, in particular for use for the protection of a human host, against diphtheria, tetanus and against a condition causally related to pertussis infection, and optionally against hepatitis B, poliomyelitis and/or disease caused by Haemophilus influenza type b.

When the immunogenic composition is an acellular immunogenic composition containing antigens of the Bordetella sp., it is for use in active immunization of a human host, in particular for use for the protection of a human host, against a condition causally related to pertussis infection. As mentioned above the protection achieved against a condition causally related to the infection by a Bordetella strain may be protection against whooping cough but also against various conditions causally related to the infection of the host by a Bordetella sp., in particular by Bordetella pertussis, such as protection against the persistence of the bacteria, against the transmission of the bacteria by the host to another individual or protection against the colonization of the airways of the infected host by the bacteria.

The invention also relates to a method of administration of the immunogenic composition to a human host, for active immunization for protection against the pathogens providing the antigens of said composition or for protection against a condition causally related to the infection by such pathogen(s).

Due to its properties resulting from the association of various antigens including the CyaA-derived polypeptide, the immunogenic composition may be used in various administration schemes corresponding to various requirements of protection of the host which are presently not fulfilled by the available products.

In a particular embodiment, the immunogenic composition or the medicinal composition disclosed herein may be for use as a dose for administration after a first dose of a vaccine composition, in particular after a first dose of an immunogenic composition a first dose which does not comprise the CyaA-derived polypeptide described herein. Doses administered after the first one are either priming doses of a multiple-dose setting wherein said further priming doses are usually administered a few weeks or less than 6 months after the first or after the immediately preceding dose. Doses administered after the first one may alternatively or also be boosting dose(s) i.e., doses usually administered more than 6 months or up to several years after the first dose or the last dose of the multiple priming doses.

In a particular embodiment, the first dose which is different from the dose comprising the immunogenic composition of the invention is different in its antigenic contents and especially does not contain a non-toxic CyaA derived polypeptide and in particular does not contain a CyaA-derived polypeptide as described herein. Such different first dose may also be different in antigen concentration, in particular it may comprise higher concentrations of the antigens than the immunogenic composition of the invention.

In particular, the immunogenic composition or the medicinal composition disclosed herein may be for use as a dose for administration after a first dose, in particular (i) as a second or as a further dose of a multiple-dose setting or (ii) as a booster dose in individuals who previously received a first vaccination dose or primary vaccination with a monovalent or a combination vaccine against at least one of the diseases selected in the group of tetanus, diphtheria and a condition causally related to infection by a Bordetella strain, in particular Bordetella pertussis, and optionally hepatitis B, poliomyelitis and disease caused by Haemophilus influenza type b.

In a particular embodiment, a booster dose is different in antigenic composition and optionally in antigen concentration from the first or from the previously administered dose(s) (previous doses), in particular because said first or previous dose(s) is(are) devoid of non-toxic CyaA-derived polypeptide as described herein in particular is(are) devoid of any form (native or enzymatically inactive or detoxified forms) of CyaA polypeptide. By contrast, said previous doses comprise of consist of one or more antigens of a Bordetella sp. selected among Pertussis toxoid (PT), Filamentous Haemagglutinin (FHA), Pertactin (PRN) and Fimbriae (Fim), in particular the Pertussis toxoid (PT), Filamentous Haemagglutinin (FHA) and Pertactin (PRN) antigens.

In a particular embodiment, a booster dose may be indicated for use as a booster dose in a prime/boost vaccination schedule wherein the host is a child above 4 years of age, an adolescent over 11 years of age or an adult, in particular an elderly person, a pregnant woman or a host who is a relative close to a pregnant woman (such as recommended in the “cocooning strategy” defined by the WHO), said host having previously received primary vaccination with a different acellular vaccine against a condition causally related to infection by Bordetella strain, in particular Bordetella pertussis either administered as a monovalent pertussis vaccine or a combination vaccine against whopping cough.

In another particular embodiment, the host is a child at birth or later, in particular until 18 months of age.

### DETAILED DESCRIPTION OF THE INVENTION

The expression “protection” applied to the immunogenic composition (or to its active ingredients eliciting an immune response) relates to and defines the ability of said immunogenic composition to elicit an immune response in a host, in particular a human host, to whom said composition has been administered for active immunization, wherein such immune response encompasses (i) a specific response to all or part of the antigens of the composition, (ii) comprises the expression of cytokines of the immune system and antibodies, in particular neutralising antibodies. As a consequence an immune response is effective against infection by the pathogen(s) providing the antigens of the composition or against a condition causally related to this pathogen. In a particular embodiment of the invention, the protective response is a preventive response such as one enabled by prophylactic vaccines. In another embodiment of the invention, the protection if therapeutic, i.e., it alleviates or prevents the pathological outcomes of the infection. Accordingly, the elicited immune response prevents the onset or the development of the disease or condition caused by the pathogen(s) against which the active immunization is sought, in particular prevents invasive stage of the pathogen or prevents its transmission to other hosts or prevents at least clinical symptoms of the disease from developing. Advantageously, the protection may also be prophylactic vaccination of the host.

The indication of the immunogenic composition to provide protection encompasses an indication to provide protection against a condition causally related to infection of a human host by a Bordetella strain, in particular B. pertussis or against other strains of Bordetella such as Bordetella parapertussis, Bordetella bronchiseptica or Bordetella hinzii. The ability to elicit protection, in particular preventive protection, is based on the demonstration of immunogenicity of the combination of the antigens originating from Bordetella contained in the composition and on the comparison of the response elicited using said antigens with that raised with conventional acellular vaccines (designated aP) against whooping cough or with whole cell vaccines (designated WCV) against whooping cough. In particular, the immune response triggered by the immune composition against Bordetella, in particular against B. pertussis encompasses a TH1- and/or TH17-oriented immune response favourable to long duration protection of the individual.

A particular aspect of the protection combination is that, in addition to protection against the whooping cough, the protection triggered by the antigens of Bordetella may extend to prevention of the invasive infection, prevention of the transmission or colonization of the airways of the host by the bacteria. The capability of the immunogenic composition to trigger a response which is TH1- and/or TH2-oriented may be a factor of the ability of the composition to extend the immune response beyond prevention of whooping cough as emphasized above.

Particular embodiments of the CyaA-derived polypeptide used in order to carry out the invention are described hereafter with respect to the structure of the polypeptide.

As mentioned above, the CyaA-derived polypeptide may be defined with respect to the sequence of SEQ ID NO: 2 which represents the amino acid sequence of the wild type CyaA protein of Bordetella pertussis. In addition, a polynucleotide encoding the CyaA as set forth in SEQ ID NO: 2, is as set forth in SEQ ID NO:1 and may be used in order to define fragments that express the CyaA-derived polypeptide used in the immunogenic composition of the invention. Another particular embodiment of a polynucleotide, encoding the CyaA as set forth in SEQ ID NO: 2, is a modified version of sequence SEQ ID NO:1, such as a sequence obtained by silent nucleotide mutations, i.e., by modifications which do not result in a change to the amino acid of SEQ ID NO:2. A particular modified version of SEQ ID NO:1 is a sequence, optimized for in expression E. coli, as set forth in SEQ ID NO:24.

The resulting CyaA-derived polypeptide of the invention which may be obtained from a polynucleotide disclosed herein comprises or consists of two segments of the native CyaA, and in a particular embodiment may accordingly be the expression of two fragments, fused together or recombined, originating from the same Bordetella CyaA protein, preferably from Bordetella pertussis. By “fragment”, it is meant a stretch or a concatenation of consecutive amino acid residues found in the sequence of the wild type Bordetella CyaA protein. The expression in a production cell of the fused fragments originating from native CyaA is obtained using a polynucleotide encoding their sequence in association with a polynucleotide comprising the coding sequence of the cyaC gene of Bordetella to enable post translational modification of the expressed amino acid sequence.

Accordingly, a “CyaA-derived polypeptide” encompasses the expressed amino acid sequence of the fused segments derived from CyaA and also such product which may have undergone post translational modifications. The thus obtained amino acid sequence provides a polypeptide derived by deletion of a continuous segment of amino acid residues (encompassing in particular or consisting of the segment between amino acid residues 227 and 321) from the original CyaA protein such as the CyaA of SEQ ID No.2. The expression also relates to variants of the CyaA-derived polypeptide of B. pertussis, in particular variants of the CyaA-derived polypeptide obtained from the amino acid sequence of SEQ ID No.2. Thus, advantageously, said “CyaA-derived polypeptide” used to carry out the invention is modified by post-translational acylation of at least one of its residues, in particular at least one of the two, preferably the two, lysine residues corresponding to the residues located in positions 860 and 983 of the full length sequence of B. pertussis, B. hinzii or B parapertussis CyaA or corresponding to the residues located in positions 859 and 982 of the full length sequence of B. bronchiseptica CyaA. By “acylation”, it is meant herein palmitoylation, i.e., addition of palmitate and/or palmitoleate group(s) on residue(s) of the CyaA-derived polypeptide used for the invention. Thus, said “CyaA-derived polypeptide” bears a palmitoyl group on some of these residues, preferably on one of the two, or the two, lysine residues corresponding to the residues 860 and 983 of the full length sequence of B. pertussis, B. hinzii or B parapertussis CyaA or corresponding to the residues located in positions 859 and 982 of the full length sequence of B. bronchiseptica CyaA. By “corresponding to”, it is meant that the residue(s) which is (are) post-translationally modified in the CyaA-derived polypeptide of the invention is (are) the one(s) the position of which matches the lysines 860 and 983 in the sequence of CyaA of B. pertussis, B. hinzii or B parapertussis CyaA (SEQ ID NO:2, 4 and 6 respectively) or the lysines 859 and 982 in the sequence of B. bronchiseptica CyaA (SEQ ID NO:8). The identification of these lysine residues in the proteins of the invention can be carried out by the person skilled in the art, by aligning and comparing the sequence of the proteins of the invention with the sequence as defined in SEQ ID NO:2, 4, 6 or 8.

The process of palmitoylation is well known from the person skilled in the art and is mediated by the cyaC gene of a Bordetella species, preferably of the Bordetella pertussis CyaC coding sequence, the natural sequence of which is set forth in SEQ ID NO:21. A version of the CyaC coding sequence, optimized for production in E. coli, is set forth in SEQ ID NO:22. This (these) post-translational modification(s) may be obtained by co-expression of the polynucleotide encoding CyaA protein, the polynucleotide encoding the CyaA-derived polypeptide of the invention and of the cyaC gene.

According to the definition provided above of the CyaA-derived polypeptide, the first segment or fragment (located in the N-terminal portion of the CyaA-derived polypeptide) begins with the first residue of SEQ ID NO:2 and ends with a residue located from position 183 to position 227 of SEQ ID NO:2.

This first segment or fragment has a size ranging from 183 to 227 residues, i.e., is at least 183 residues in length and is at most 227 residues in length. In a particular embodiment, this segment or fragment is at least 183, at least 190, at least 200, at least 210 or at least 220 residues in length. In a particular embodiment, the size of this first segment or fragment is 183 residues or is 227 residues.

Thus, this segment or fragment begins with the first residue of SEQ ID NO:2 and ends with a residue selected from the group consisting of residues 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226 and 227 of SEQ ID NO: 2.

In other words, this first segment or fragment comprises or consists of a sequence which is selected from the group consisting of residues 1-183, 1-184, 1-185, 1-186, 1-187, 1-188, 1-189, 1-190, 1-191, 1-192, 1-193, 1-194, 1-195, 1-196, 1-197, 1-198, 1-199, 1-200, 1-201, 1-202, 1-203, 1-204, 1-205, 1-206, 1-207, 1-208, 1-209, 1-210, 1-211, 1-212, 1-213, 1-214, 1-215, 1-216, 1-217, 1-218, 1-219, 1-220, 1-221, 1-222, 1-223, 1-224, 1-225, 1-226 and 1-227 of SEQ ID NO: 2.

In a particular embodiment, this first segment or fragment comprises or consists of residues 1 to 227 of SEQ ID NO:2 or of residues 1 to 183 of SEQ ID NO:2.

In a particular embodiment, the polynucleotide encoding said first segment or fragment begins with the first nucleotide of SEQ ID NO:1 or of SEQ ID NO:24 and ends with a nucleotide located from position 549 to position 681 of SEQ ID NO:1 or of SEQ ID NO:24, provided that the length of said polynucleotide segment or fragment is a multiple of 3. Thus, the polynucleotide encoding this segment or fragment comprises or consists of a sequence which is selected from the group consisting of residues 1-549, 1-552, 1-555, 1-558, 1-561, 1-564, 1-567, 1-570, 1-573, 1-576, 1-579, 1-582, 1-585, 1-588, 1-591, 1-594, 1-597, 1-600, 1-603, 1-606, 1-609, 1-612, 1-615, 1-618, 1-621, 1-624, 1-627, 1-630, 1-633, 1-636, 1-639, 1-642, 1-645, 1-648, 1-651, 1-654, 1-657, 1-660, 1-663, 1-666, 1-669, 1-672, 1-675, 1-678 and 1-681 of SEQ ID NO: 1 or of SEQ ID NO:24.

The second segment or fragment (the C-terminal portion of the CyaA-derived polypeptide) begins with a residue located from position 321 to position 387 of SEQ ID NO:2 and ends with the last residue of SEQ ID NO:2.

This second segment or fragment has a size ranging from 1320 to 1386 residues, i.e., is at least 1320 residues and is at most 1386 residues in length. In a particular embodiment, this segment or fragment is at least 1320, at least 1330, at least 1340, at least 1350, at least 1360, at least 1370 or at least 1380. In a particular embodiment, the size of this second segment or fragment is 1320 residues or is 1386 residues.

Thus, this second segment or fragment begins with a residue selected from the group consisting of residues 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386 and 387 of SEQ ID NO:2 and ends with the last residue (i.e., residue 1706) of SEQ ID NO:2.

In other words, this second segment or fragment comprises or consists of a sequence which is selected from the group consisting of residues 321-1706, 322-1706, 323-1706, 324-1706, 325-1706, 326-1706, 327-1706, 328-1706, 329-1706, 330-1706, 331-1706, 332-1706, 333-1706, 334-1706, 335-1706, 336-1706, 337-1706, 338-1706, 339-1706, 340-1706, 341-1706, 342-1706, 343-1706, 344-1706, 345-1706, 346-1706, 347-1706, 348-1706, 349-1706, 350-1706, 351-1706, 352-1706, 353-1706, 354-1706, 355-1706, 356-1706, 357-1706, 358-1706, 359-1706, 360-1706, 361-1706, 362-1706, 363-1706, 364-1706, 365-1706, 366-1706, 367-1706, 368-1706, 369-1706, 370-1706, 371-1706, 372-1706, 373-1706, 374-1706, 375-1706, 376-1706, 377-1706, 378-1706, 379-1706, 380-1706, 381-1706, 382-1706, 383-1706, 384-1706, 385-1706, 386-1706 and 387-1706 of SEQ ID NO:2

In a particular embodiment, this second segment or fragment comprises or consists of residues 321-1706 of SEQ ID NO:2 or of residues 387-1076 of SEQ ID NO:2.

In a particular embodiment, the polynucleotide encoding said second segment or fragment begins with a nucleotide located from position 961 to position 1159 of SEQ ID NO:1 or of SEQ ID NO:24 and ends with the last nucleotide (i.e., nucleotide 5118) of SEQ ID NO:1 or of SEQ ID NO:24, provided that the length of said nucleotide segment or fragment is a multiple of 3. Thus, the polynucleotide encoding this second segment or fragment comprises or consist of a sequence which is selected from the group consisting of residues 961-5118, 964-5118, 967-5118, 970-5118, 973-5118, 976-5118, 979-5118, 982-5118, 985-5118, 988-5118, 991-5118, 994-5118, 997-5118, 1000-5118, 1003-5118, 1006-5118, 1009-5118, 1012-5118, 1015-5118, 1018-5118, 1021-5118, 1024-5118, 1027-5118, 1030-5118, 1033-5118, 1036-5118, 1039-5118, 1042-5118, 1045-5118, 1048-5118, 1051-5118, 1054-5118, 1057-5118, 1060-5118, 1063-5118, 1066-5118, 1069-5118, 1072-5118, 1075-5118, 1078-5118, 1081-5118, 1084-5118, 1087-5118, 1090-5118, 1093-5118, 1096-5118, 1099-5118, 1102-5118, 1105-5118, 1108-5118, 1111-5118, 1114-5118, 1117-5118, 1120-5118, 1123-5118, 1126-5118, 1129-5118, 1132-5118, 1135-5118, 1138-5118, 1141-5118, 1144-5118, 1147-5118, 1150-5118, 1153-5118, 1156-5118 and 1159-5118 of SEQ ID NO:1 or of SEQ ID NO:69.

In a particular embodiment, the CyaA-derived polypeptide comprises or consists of a polypeptide of the sequence as set forth in SEQ ID NO:10; SEQ ID NO:10 consisting of a segment or fragment consisting of residues 1 to 227 of SEQ ID NO:2 fused to a segment or fragment consisting of residues 321 to 1706 of SEQ ID NO:2.

In another particular embodiment, the CyaA-derived polypeptide comprises or consists of a polypeptide of the sequence as set forth in SEQ ID NO:12; SEQ ID NO:12 consisting of a segment or fragment consisting of residues 1 to 183 of SEQ ID NO:2 fused to a segment or fragment consisting of residues 387 to 1706 of SEQ ID NO:2.

Other particular embodiments are also disclosed:
the CyaA-derived polypeptide comprises or consists of a polypeptide of the sequence as set forth in SEQ ID NO:19, i.e., a sequence consists of a segment or fragment consisting of residues 1 to 227 of SEQ ID NO:2 fused to a segment or fragment consisting of residues 387 to 1706 of SEQ ID NO:2, and
the CyaA-derived polypeptide comprises or consists of a polypeptide of the sequence as set forth in SEQ ID NO:20, i.e., a sequence consisting of a segment or fragment consisting of residues 1 to 183 of SEQ ID NO:2 fused to a segment or fragment consisting of residues 321 to 1706 of SEQ ID NO:2.

The expression “fused to” when reference is made to a protein or a polypeptide means that the peptide segments or fragments (e.g., several CyaA segments or fragments) are covalently linked together by a peptide bond. The order of these different peptide parts is described herein as from N-terminal to C-terminal, i.e., the last C-terminal residue of a first part is linked to the first N-residue of the other part by a peptide bond. The expression “fused to” when reference is made to a polynucleotide, means that two or more polynucleotide parts (e.g., several nucleotide CyaA segments or fragments) are covalently linked together by a phosphodiester bond. The order of these different nucleotide parts is described herein as from 5′ to 3′, i.e., the last 3′ nucleotide of a first part is linked to the first 5′ nucleotide of the other part by a phosphodiester bond. The polynucleotide consisting of the fusion of nucleotide sequences is in particular obtained as a recombinant polynucleotide, including by deletion of nucleotides in the native coding sequence of cyaA which are between the retained segments. The CyaA-derived polypeptide may be obtained as a result of expression in cells such as bacteria, in particular E. coli and the polynucleotide expressing the polypeptide may be obtained by genetic recombination or by synthesis of the sequence using the information of the polynucleotide encoding the CyaA-derived polypeptide.

The invention also concerns a polynucleotide encoding a variant CyaA-derived polypeptide, wherein said first segment or fragment is a polypeptide with at least 95% identity with a determined segment or fragment of the Bordetella pertussis CyaA protein as set forth in SEQ ID NO: 2, the sequence of said segment or fragment beginning with the first residue of SEQ ID NO:2 and ending with a residue located from position 183 to position 227 of SEQ ID NO:2, and/or wherein said second segment or fragment is a variant with at least 95% identity with a segment or fragment beginning with a residue located from position 321 to position 387 of SEQ ID NO:2 and ending with the last residue of SEQ ID NO:2.

By “a variant with at least 95% identity” when reference is made to a protein or a polypeptide, it is meant a protein sequence whose amino acid identity is at least 95%, at least 96%, at least 97%, at least 98% or at least 99% with the polypeptide from which it varies. The percentage of identity is calculated, comparing the full-length sequence of both said variant and said polypeptide of reference, in particular over the shorter of the two sequences. Thus, a variant has 95% of identity with a polypeptide, when 5% of its residues differ from the residues of this polypeptide, by one or more addition(s) and/or one or more deletion(s) and/or one or more substitution(s). In a particular embodiment, said variant differs from said polypeptide only by substitutions, preferably conservative substitutions and accordingly it keeps the same length as the sequence from which it varies. In another embodiment, said variant differs from said polypeptide by at least 1 single amino-acid deletion, preferably by 1, 2, 3, 4 or 5 single amino-acid deletion(s), and by substitutions, preferably conservative substitutions.

The invention also relates to polynucleotide variants having an identity of at least 75% with the polynucleotides encoding portions (or fragments) of SEQ ID NO:1. In a particular embodiment, the polynucleotide encoding said first fragment has an identity of 75% with a polynucleotide beginning with the first nucleotide of SEQ ID NO:1 and ends with a nucleotide located from position 549 to position 681 of SEQ ID NO:1 provided that the length of said nucleotide fragment is a multiple of 3. In another embodiment, independently or in combination with the above statement, the polynucleotide encoding said second fragment has an identity of 75% with a polynucleotide beginning with a nucleotide located from position 961 to position 1159 of SEQ ID NO:1 and ends with the last nucleotide (i.e., nucleotide 5118) of SEQ ID NO:1, provided that the length of said nucleotide fragment is a multiple of 3. In a particular embodiment, the polynucleotides encoding said first and second segments or fragments originate from a polynucleotide, the full-length sequence of which has at least 75% identity with SEQ ID NO:1. An example of such variant is SEQ ID NO:24. In a particular embodiment, the polynucleotide variant results from degeneracy of the genetic code applied to the polynucleotide obtained from SEQ ID NO:1 as disclosed above or to the polynucleotide of SEQ ID NO:24. In a particular embodiment, the polynucleotide variant thus obtained has a degenerated base at the wobble position.

By “a variant with at least 75% identity” when reference is made to a polynucleotide, it is meant a nucleotide sequence whose nucleotide identity is at least 75%, at least 79%, at least 80%, at least 85%, at least 90%, at least 95%, at least 96%, at least 97%, at least 98% or at least 99% with the polynucleotide from which it varies. The percentage of identity is calculated, comparing the full-length sequence of both said variant and the polynucleotide from which it varies, in particular over the shorter of the two sequences. Thus, a variant has 75% of identity with a polynucleotide, when 25% of its nucleotides differ from the nucleotides of said polynucleotide, by one or more nucleotide addition(s) and/or one or more nucleotide deletion(s) and/or one or more nucleotide substitution(s). In a particular embodiment, said variant differs only by nucleotide substitutions, and accordingly it keeps the same length as the sequence from which it varies. In a particular embodiment, said variant differs only by nucleotide silent mutations, and accordingly it keeps encoding the same protein as the one encoded by the sequence from which it varies. In a particular embodiment, said variant differs only by nucleotide substitutions, a part of them being silent mutations, such that the sequence of the protein encoded by said polynucleotide variant has at least 95% of identity with a protein or polypeptide of the invention, or has 100% identity.

Nucleotide and protein identity percentages as indicated herein may be calculated by well-known programs based on the Needleman and Wunsch algorithm, such as MeAlign [18].

In a particular embodiment, the variant CyaA-derived polypeptide suitable for use in the invention keeps its capacity to bind to target cells and/or to translocate its adenylate cyclase (AC) domain into the cytosol of the target cells. In a particular embodiment, target cells are CD11b-expressing cells, i.e., cells that express the CD11b/CD18 receptor on their surface (CD11b⁺). In particular, these cells are granulocytes/neutrophils, macrophages, NK cells, subsets of T CD8⁺, subsets of B cells, dendritic cells such as Langerhans cells, or myeloid dendritic cells.

The capacity of the variants of the invention to bind to target cells can be assayed especially according to the methods disclosed in EP03291486 or in WO02/22169 application. Furthermore, the capacity of the variant to translocate its N-terminal domain into the cytosol of target cells can be assayed by applying the method described in WO02/22169 application, or the method detailed in example A with the p105 peptide.

Preferred variants of CyaA-derived polypeptide obtained from the protein of SEQ ID NO.2 which is the full-length wild type sequence of the Bordetella pertussis CyaA protein are obtained from CyaA proteins of other Bordetella sp. provided herein by reference to their amino acid sequence as set forth in SEQ ID NO: 4 (CyaA protein of Bordetella hinzii), SEQ ID NO: 6 (CyaA protein of Bordetella parapertussis) and SEQ ID NO: 8 (CyaA protein of Bordetella bronchiseptica). The nucleotide sequence, encoding SEQ ID NOs: 4, 6 and 8, is as set forth in SEQ ID NOs:3, 5 and 7 respectively or is a variant of SEQ ID NOs:3, 5 and 7 by silent mutations. Accordingly, CyaA-derived polypeptides to carry out the present invention, which are variants of the polypeptide obtained by reference to the sequence of SEQ ID NO.2 are segments or fragments fused as disclosed herein which are obtained from the amino acid sequences of Bordetella parapertussis, Bordetella bronchiseptica or Bordetella hinzii and defined by alignment of these sequences with the segments defined in SEQ ID NO.2 for the purpose of the invention.

The CyaA-derived polypeptide defined in order to carry out the invention does not comprise or does not consist of SEQ ID NOs: 2, 4, 6 and 8. Moreover, a polynucleotide encoding a variant CyaA-derived polypeptide suitable to carry out the invention does not comprise or does not consist of SEQ ID NOs: 3, 5 or 7.

Particular CyaA-derived polypeptides which are suitable to carry out the invention and which result from the fusion of 2 segments or fragments of Bordetella pertussis CyaA according to the definition provided and their variants obtained from the fusion of the fragments equivalent to those of B. pertussis and taken from the amino acid sequence of CyaA of any of Bordetella parapertussis, Bordetella bronchiseptica or Bordetella hinzii are selected in the group of:

a polypeptide comprising or consisting of the sequence as set forth in SEQ ID NO:10 or a polypeptide variant obtained from Bordetella parapertussis, Bordetella bronchiseptica or Bordetella hinzii and characterized by an amino acid sequence matching the amino acid residue positions of SEQ ID No.10;

a polypeptide comprising or consisting of the sequence as set forth in SEQ ID NO:12 or a variant obtained from Bordetella parapertussis, Bordetella bronchiseptica or Bordetella hinzii, and characterized by an amino acid sequence matching the amino acid residue positions of SEQ ID No.12,

a polypeptide comprising or consisting of the sequence as set forth in SEQ ID NO:19 or a variant obtained from Bordetella parapertussis, Bordetella bronchiseptica or Bordetella hinzii, and characterized by an amino acid sequence matching the amino acid residue positions of SEQ ID No.19,

a polypeptide comprising or consisting of the sequence as set forth in SEQ ID NO:20 or a variant obtained from Bordetella parapertussis, Bordetella bronchiseptica or Bordetella hinzii and characterized by an amino acid sequence matching the amino acid residue positions of SEQ ID No.20.

Polynucleotides and Preparation of the CyaA-Derived Polypeptides

In a particular embodiment of the invention, a polynucleotide is used that encodes a variant CyaA-derived polypeptide, preferably as variant of a B. pertussis CyaA-derived polypeptide as defined herein, and such polynucleotide encodes a polypeptide comprising or consisting of:

(a) a segment or a fragment of the Bordetella CyaA protein as set forth in SEQ ID NO: 4, 6 or 8, the sequence of said segment of fragment beginning with the first residue of SEQ ID NO:4, 6 or 8 and ending with a residue located from position 183 to position 227 of SEQ ID NO:4, 6 or 8, fused to

(b) a segment or a fragment of the Bordetella CyaA protein as set forth respectively in SEQ ID NO: 4, 6 or 8, the sequence of said segment or fragment beginning with a residue located from position 321 to position 387 of SEQ ID NO: 4, 6 or 8 and ending with the last residue of SEQ ID NO: 4, 6 or 8.

The definitions given above for the particular CyaA-derived polypeptide comprising fragments of SEQ ID NO:2 apply identically to the variant CyaA-derived polypeptide comprising fragments of SEQ ID NO:4 and 6.

Regarding the variant CyaA-derived polypeptide comprising fragments of SEQ ID NO:8, all definitions apply identically, with the exception of the last residue of SEQ ID NO:8 is residue 1705 instead of residue 1706. Therefore, for the variant CyaA-derived polypeptide comprising segments or fragments of SEQ ID NO:8, all aspects referring to residue 1706 must be replaced by residue 1705. In particular, the second segment or fragment has a size ranging from 1319 to 1385 residues, and is preferably 1319 residues or 1385 residues in length. Regarding a polynucleotide encoding variant CyaA-derived polypeptide comprising segments or fragments of SEQ ID NO: 8, all definitions and embodiments referring to nucleotide 5118 must be replaced by nucleotide 5115.

Particular polynucleotides encoding variant CyaA-derived polypeptides suitable to carry out the invention are selected among the following polynucleotide consisting of:

1) a polynucleotide encoding the polypeptide as set forth in SEQ ID NO:13; SEQ ID NO:13 consists of a segment or a fragment consisting of residues 1 to 227 of SEQ ID NO:4 fused to a segment or a fragment consisting of residues 321 to 1706 of SEQ ID NO:4;

2) a polynucleotide encoding the polypeptide as set forth in SEQ ID NO:14; SEQ ID NO:14 consists of a segment or a fragment consisting of residues 1 to 183 of SEQ ID NO:4 fused to a segment or a fragment consisting of residues 387 to 1706 of SEQ ID NO:4;

3) a polynucleotide encoding the polypeptide as set forth in SEQ ID NO:15; SEQ ID NO:15 consists of a segment or a fragment consisting of residues 1 to 227 of SEQ ID NO:6 fused to a segment or a fragment consisting of residues 321 to 1706 of SEQ ID NO:6;

4) a polynucleotide encoding the polypeptide as set forth in SEQ ID NO:16; SEQ ID NO:16 consists of a segment or a fragment consisting of residues 1 to 183 of SEQ ID NO:6 fused to a segment or a fragment consisting of residues 387 to 1706 of SEQ ID NO:6;

5) a polynucleotide encoding the polypeptide as set forth in SEQ ID NO:17; SEQ ID NO:17 consists of a segment or a fragment consisting of residues 1 to 227 of SEQ ID NO:8 fused to a segment or a fragment consisting of residues 321 to 1705 of SEQ ID NO:8; and

6) a polynucleotide encoding the polypeptide as set forth in SEQ ID NO:18; SEQ ID NO:18 consists of a segment or a fragment consisting of residues 1 to 183 of SEQ ID NO:8 fused to a segment or a fragment consisting of residues 387 to 1705 of SEQ ID NO:8.

In a particular embodiment, the polynucleotide encoding the CyaA-derived polypeptide or the variant CyaA-derived polypeptide used in the invention may hence be defined as a deleted version of the full-length Bordetella CyaA coding nucleotide sequence, i.e., a polynucleotide encoding a polypeptide consisting of a deletion fragment of SEQ ID NO:2, 4, 6 or 8 wherein the deletion encompasses a segment of the native sequence covering an amino acid segment the first amino acid residue of which is located from residue 184 to residue 228 of SEQ ID NO: 2, 4, 6 or 8 respectively, and the last amino acid residue of which is located from residue 320 to residue 386 of SEQ ID NO: 2, 4, 6 or 8 respectively. In a particular embodiment, said polynucleotide encodes a polypeptide consisting of a deleted version of SEQ ID NO:2, 4, 6 or 8, which is deleted for a polynucleotide encoding a polypeptide fragment whose first amino acid residue is selected from the group consisting of residues 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227 and 228 of SEQ ID NO: 2, 4, 6 or 8 respectively, and whose last amino acid residue is selected from the group consisting of residues 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385 or 386 of SEQ ID NO: 2, 4, 6 or 8 respectively.

A method to produce a polynucleotide encoding the CyaA-derived polypeptide in particular a variant CyaA-derived polypeptide as described herein is also part of the invention. This method comprises the steps of (a) deleting, from a polynucleotide encoding the Bordetella CyaA as set forth in SEQ ID NO: 2, 4, 6 or 8, a nucleotide fragment of consecutive nucleotide residues in said sequences, the first three nucleotides of which encode an amino acid residue located from residue 184 to residue 228 of SEQ ID NO: 2, 4, 6 or 8, and the last three nucleotides of which encode an amino acid residue located from residue 320 to residue 386 of SEQ ID NO: 2, 4, 6 or 8; and (b) recovering said polynucleotide.

Alternatively, the polynucleotide encoding the CyaA-derived polypeptide is chemically synthesized, using conventional methods, according to the sought CyaA-derived polypeptide sequence, and optionally taking into account the degeneracy of the genetic code and/or the optimization of expression.

The invention also uses the CyaA-derived polypeptides encoded by the polynucleotides thus defined. Particular CyaA-derived polypeptides consist of a sequence as set forth in SEQ ID NO: 10, 12, 13, 14, 15, 16, 17, 18, 19 and 20.

A polynucleotide encoding a CyaA-derived polypeptide, including a variant CyaA-derived polypeptide is used to produce in a cell, in particular in a bacterial cell, especially in E. coli, a polynucleotide encoding said CyaA-derived polypeptide, i.e., a B. pertussis CyaA-derived polypeptide or a variant CyaA-derived polypeptide thereof.

A method to produce a polynucleotide encoding a CyaA-derived polypeptide, comprises:

(a) deleting, from a polynucleotide encoding the Bordetella CyaA as set forth in SEQ ID NO: 2, 4, 6 or 8 or encoding a variant with at least 95% identity with SEQ ID NO: 2, a nucleotide segment or fragment, the first three nucleotides of which encode an amino acid residue located from residue 184 to residue 228 of SEQ ID NO: 2, 4, 6 or 8, whose the last 3 nucleotides of which encode an amino acid residue located from residue 320 to residue 386 of SEQ ID NO: 2, 4, 6 or 8; and

(b) recovering said polynucleotide encoding a CyaA-derived polypeptide.

A method to produce a CyaA-derived polypeptide comprises:

(a) deleting, from a polynucleotide encoding the Bordetella CyaA as set forth in SEQ ID NO: 2, 4, 6 or 8 or encoding a variant with at least 95% identity with SEQ ID NO: 2, a nucleotide fragment, the first three nucleotides of which encode an amino acid residue located from residue 184 to residue 228 of SEQ ID NO: 2, 4, 6 or 8, whose the last 3 nucleotides of which encode an amino acid residue located to from residue 320 to residue 386 of SEQ ID NO: 2, 4, 6 or 8;

(b) expressing, in a cell, especially in a bacterial cell and in particular in E. coli, the polynucleotide obtained in (a); and

(c) recovering said expressed CyaA-derived polypeptide.

The method to produce the CyaA-derived polypeptide may further comprise the step of combining in the polynucleotide construct thus obtained and a polynucleotide encoding the CyaC protein.

In a particular embodiment the CyaA-derived polypeptide is expressed in such cells using the plasmid pGTP-gtCyaAd93-CyaCopt which has the sequence of SEQ ID NO.25

Within the present invention, when reference is made to the “first three nucleotides” or the “last three nucleotides”, it is understood that these three nucleotides refer to a codon which corresponds, according to the genetic code, to an amino acid residue identified by its position in SEQ ID NO: 2, 4, 6 or 8. Thus, the size of the polynucleotide nucleotide deletion is preferably a multiple of 3. Moreover, in addition to be a multiple of 3 in size, the polynucleotide nucleotide deletion is in frame, i.e., the deletion removes the sought amino acid residues, without modifying the reading frame, nor modifying the residues surrounding (upstream and downstream) the deletion.

The preparation of the CyaA-derived polypeptide as an expression product of a polynucleotide encoding same is described in particular in WO 2014/016310 and may be achieved through the use of techniques well known to the person skilled in the art.

In a particular embodiment, the polynucleotide construct expressing the CyaA-derived polypeptide and CyaC protein comprises from 5′ end to 3′ end, the cyaA polynucleotide or gene, advantageously consisting of an optimized sequence for expression in a determined host or production cell, e.g. E. Coli and the cyaC polynucleotide or gene, advantageously consisting of an optimized sequence for expression in a determined host or production cell, e.g. E. coli. This order of the insertion of the polynucleotides in the construct favours the expression of the CyaA-derived polypeptide and CyaC protein in respective quantities and conformation suitable for increasing efficiency of expression of post translational modified version of CyaA.

In a particular embodiment, except for the deletion of the segment or fragment [whose first amino acid residue is located from residue 184 to residue 228 of SEQ ID NO: 2, 4, 6 or 8 respectively, and whose last amino acid residue is located from residue 320 to residue 386 of SEQ ID NO: 2, 4, 6 or 8 respectively] carried out in the wild type Bordetella CyaA protein disclosed herein, the CyaA-derived polypeptide suitable to carry out the invention does not undergo any other variation (addition, deletion and/or substitution) as compared to SEQ ID NO:2, 4, 6 or 8.

As a consequence of its amino acid sequence, the CyaA derived protein is non-toxic, in particular non-cytotoxic, i.e., its enzymatic activity adenylate cyclase has been inactivated as a result of the deletion of the segment or fragment whose first amino acid residue is located from residue 184 to residue 228 of SEQ ID NO: 2, 4, 6 or 8 respectively, and whose last amino acid residue is located from residue 320 to residue 386 of SEQ ID NO: 2, 4, 6 or 8 respectively (deletion of a segment or fragment is accordingly deletion of at least a fragment of 93 amino residues (designated “d93”)). Therefore, in a particular embodiment, no insertion, deletion or substitution has been carried out. In particular, when the residues 188 and 189 of CyaA are still present in the proteins of the invention, no dipeptide (such as the dipeptide LQ or GS) is inserted between the residues 188 and 189.

Additional features and properties of the embodiments of the invention will be apparent in the Examples and Figures which follow.

### BRIEF DESCRIPTION OF THE FIGURES

FIGS. 1A-B:

Cytokine responses after vaccination with Tdap+/−GTL003: Intramuscular route The results in histograms are representative of mean recall response of 4 animals. The splenocytes of 4 animals were pooled together and were cultured in duplicates. Each result is a mean response of duplicate wells. Levels of cytokines were analyzed using multiplex bead based ELISA and % variation (CV) between duplicates were below 20%. Error bars representative of % CV of duplicate wells.

FIG. 1A:

Cytokine response of animals primed with Tdap and boosted either with Tdap or with Tdap+GTL003

White bars: Tdap prime+Tdap boost

Grey bars: Tdap prime+Tdap+GTL003 boost

FIG. 1B:

Cytokine response of animals primed with Tdap and boosted either with WCV or with Tdap+GTL003

White bars: Tdap prime+tdap+GTL003 boost

Grey bars: Tdap prime+WCV boost

FIGS. 2A-B:

Cytokine responses after vaccination with Tdap+/−GTL003: Intraperitoneal route

The results in histograms are representative of mean recall response of 4 animals. The splenocytes of 4 animals were pooled together and were cultured in duplicates. Each result is a mean response of duplicate wells. Levels of cytokines were analyzed using multiplex bead based ELISA and % variation (CV) between duplicates were below 20%. Error bars representative of % CV of duplicate wells.

FIG. 2A:

Cytokine response of animals primed with Tdap and boosted either with Tdap or with Tdap+GTL003

White bars: Tdap prime+Tdap boost

Grey bars: Tdap prime+tdap+GTL003 boost

FIG. 2B:

Cytokine response of animals primed with Tdap and boosted either with WCV or with Tdap+GTL003

White bars: Tdap prime+tdap+GTL003 boost

Grey bars: Tdap prime+WCV boost

FIG. 3:

Cytokine responses after immunization with Tdap or Tdap+GTL00: intraperitoneal INF-g immune response measured in animals vaccinated either with adjuvanted-Tdap alone or with adjuvanted-Tdap+GTL003

In vitro restimulations were performed with antigens contained in the Tdap or antigens contained in the Tdap+GTL003

FIG. 4:

Cytokine responses after immunization with Tdap or Tdap+GTL00: intraperitoneal IL-17 immune response measured in animals vaccinated either with adjuvanted-Tdap alone or with adjuvanted-Tdap+GTL003

FIGS. 5A-D:

Isotyping Results (from intramuscular vaccination)
Results are from two pools of two animals each
Histograms represent mean GMT
Error bars represent 95% CI of these titers
Pool to Pool variation observed as expected
In IM route, the effects were mixed Th1/Th2

FIG. 5A:

Pertussis toxin-IgG1 antibodies after a prime with Tdap and a boost with either Tdap, Tdap+GTL003 or WCV

FIG. 5B:

Pertussis toxin-IgG2a antibodies after a prime with Tdap and a boost with either Tdap, Tdap+GTL003 or WCV

FIG. 5C:

Pertussis toxin-IgG2b antibodies after a prime with Tdap and a boost with either Tdap, Tdap+GTL003 or WCV

FIG. 5D: Pertussis toxin-IgG3 antibodies after a prime with Tdap and a boost with either Tdap, Tdap+GTL003 or WCV

FIGS. 6A-D:

Isotyping Results (from intramuscular vaccination)
Results are from two pools of two animals each
Histograms represent mean GMT
Error bars represent 95% CI of these titers
Pool to Pool variation observed as expected
In IM route, the effects were mixed Th1/Th2

FIG. 6A:

FHA-IgG1 antibodies after a prime with Tdap and a boost with either Tdap, Tdap+GTL003 or WCV

FIG. 6B:

FHA-IgG2a antibodies after a prime with Tdap and a boost with either Tdap, Tdap+GTL003 or WCV

FIG. 6C:

FHA-IgG2b antibodies after a prime with Tdap and a boost with either Tdap, Tdap+GTL003 or WCV

FIG. 6D:

FHA-IgG3 antibodies after a prime with Tdap and a boost with either Tdap, Tdap+GTL003 or WCV

FIG. 7:

Plasmid maps of GTL003 (gtCyaad93)

FIG. 8:

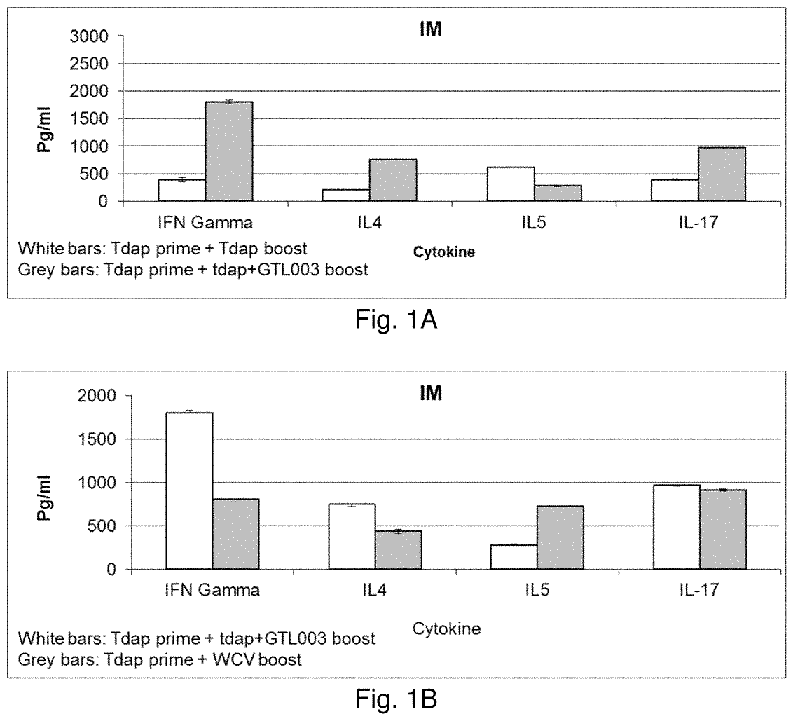

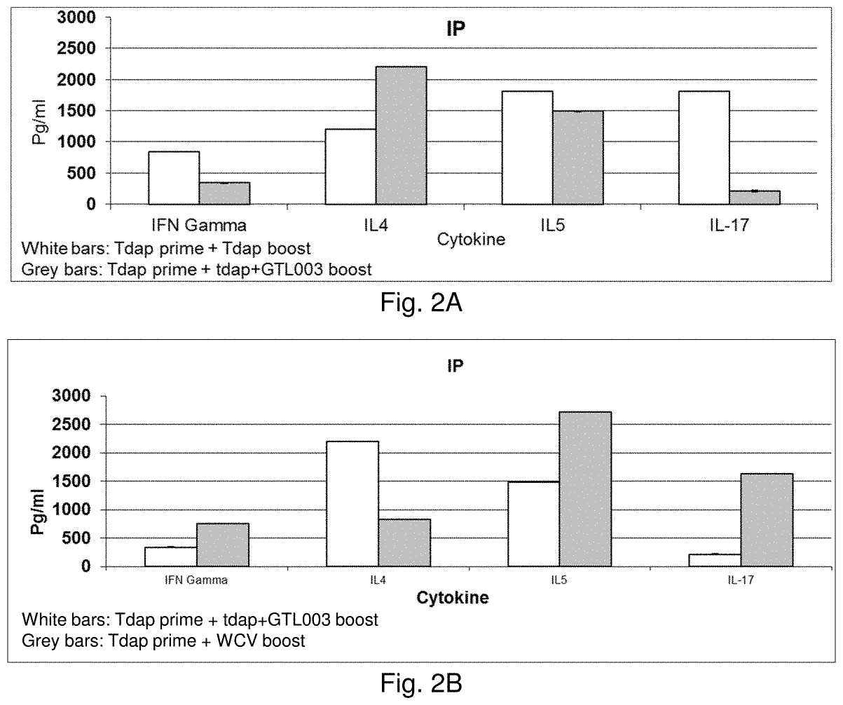

Plasmid sequence (SEQ ID No.25)

### EXAMPLES

### Abbreviations

| | |
|---|---|
| aa | Aminoacid |
| ACT | GTL003 |
| aP | Acellular pertussis vaccine |
| APC | Antigen-presenting cells |
| CD | Cluster designation |
| CTL | Cytotoxic T lymphocytes |
| CyaA | Adenylate cyclase |
| DC | Dendritic cells |
| DTaP | Diphtheria and tetanus toxoids combined with |
| | acellular pertussis vaccine |
| DTwP | Diphteria and tetanus toxoids combined with whole |
| | cell pertussis inactivated bacteria vaccine |
| FHA | Filamentous hemagglutinin |
| FIM | Fimbriae |
| GTL003 | recombinant genetically detoxified CyaA described |
| | in WO 2014/016310 |
| id | Intradermal |
| IL-4 | Interleukin 4 |
| INF-γ | Interferon gamma |
| im | Intramuscular |
| ip | Intraperitoneal |
| KLH | Keyhole Limpet hemocyanin |
| LPS | Lipopolysaccharide |
| PRN | Pertactin |
| PT | Pertussis toxin |
| Tdap* | Tetanus and diphtheria toxoids combined with |
| | acellular pertussis reduced vaccine |
| TH1 | T-helper l cells |
| TH2 | T-helper 2 cells |
| TH17 | T-helper 17 cells |
| TLR | Toll like receptor |
| WCV | Whole cell pertussis vaccine |
| WHO | World health organization |
| wP | Whole cell pertussis vaccine |

*Tdap distinguishes over DTap vaccine in that it contains a lower concentration of antigens: Tdap indeed reflects the concentration of antigens which is used for booster doses in conventional vaccines (such as Boostrix ™-GSK Biologicals S.A)

1. Material and Methods

1.1. Mice

13-23 grams female/male NIH mice were in bred at Serum Institute of India animal house. Mice were housed under pathogen-free conditions with water and food ad libitum. Procedures involving animals and their care were conformed to SIIL's guidelines that comply with national and international laws and policies and that are reviewed by the local ethical committee.

1.2. Vaccine

Manufacturing and Formulation of Tdap Vaccine

The TdaP Vaccine is a blend of individually adsorbed antigens viz; Diphtheria Toxoid, Tetanus toxoid, Pertussis toxoid, Filamentous haemagglutinin and Pertactin. The tetanus component is purified toxoid manufactured by the chemical detoxification of toxin produced by Clostridium tetani. Tetanus toxoid is prepared from the toxin produced by the growth of this strain of Clostridium tetani in Semi-synthetic (meat free) medium using fermentation technology. The diphtheria toxoid is produced using Corynebacterium diphtheriae. Toxin is concentrated, partially purified and subsequently detoxified. And the toxoid is further purified to produce purified diphtheria toxoid.

The bulk Acellular pertussis is non-infectious and made up of sterile combination of three purified pertussis antigens i.e. Pertussis toxoid, Filamentous haemagglutinin and Pertactin. The fermentation is followed by chromatography based purifications followed by chemical detoxification of Toxoids. The Pertussis Toxin (PT) and Filamentous Haemagglutinin (FHA) are produced as extra cellular proteins which are secreted during fermentation process where as Pertactin (69 kDa outer membrane protein) is extracted from cells.

Tdap is a reduced antigen version of Acellular pertussis based DTaP vaccine. On Dec. 17, 1991 the first Acellular pertussis vaccine viz; Acel-imune by lederle was licensed as a 4th and 5th Booster vaccine. These vaccines are classical vaccines.

Construction and Purification of GTL003

The DNA sequence of wild type CyaA (CyaAwt: GeneBank: CAE41066.1) was optimized and synthetized (GeneCust) for the expression in E. coli. The optimized DNA sequence is named gtCyaA.

The gtCyaA was then inserted in the pGTPc608 plasmid that contains a pTAC inducible promoter (plasmid provided by GTP Technology, Labége, France).

The deletion of 93 aa in gtCyaA was generated between aa 227 and 321 by enzymatic restriction/ligation. The obtained amino acid sequence is derived from the sequence of SEQ ID No.2 wherein the segment from amino acid residue 227 to amino acid residue 321 has been deleted.

Purification protocol was already described in EP1 576 967 B1.

1.3. Vaccine Formulation and Administration

Three vaccines were used to vaccinate mice against pertussis. All vaccines were adjuvanted with aluminum hydroxide in buffer. WCV was not adjuvanted.

TABLE 1
Vaccine antigen and adjuvant doses injected per immunization

| | Antigen and adjuvant injected per immunization/per mouse | | | | | | |
|---|---|---|---|---|---|---|---|
| | DT | TT | PT | FHA | PRN | GTL003 | Alum |
| Vaccines | (Lf) | (Lf) | (mcg) | (mcg) | (mcg) | (mcg) | (mg) |
| Tdap(1:10) | 0.25 | 0.5 | 0.8 | 0.8 | 0.25 | — | 0.39 |
| Tdap(1:10) + | 0.25 | 0.5 | 0.8 | 0.8 | 0.25 | 25 | 0.39 |
| GTL003 | | | | | | | |

WCV used in the present experiments corresponds to 0.5 IU/dose of B. pertussis (without alum) injected in mice.

In GTL003-containing vaccines, 25 μg of GTL003 were added to the Tdap vaccines preparation before injection. In prime boost protocol, mice were vaccinated twice via the same route, i.e. im, ip or sc, at day 0 and day 28.

TABLE 2
summary of the vaccination schedule, In vitro stimulation was
performed aP antigens +GTL003

| | | | In vitro |
|---|---|---|---|
| | 1st Dose | 2nd Dose | Stimulus at |
| Groups | at day 0 | at day 28 | day 42 |
| Group 1 | Tdap (1:10) | Tdap (1:10) | aP antigens + |
| Group 2 | Tdap (1:10) | Tdap (1:10) + | GTL003 |
| | | 25 μg GTL003 | |
| Group 3 | Tdap (1:10) | WCV | |

1.4. Cytokine Response Profiling

Spleen cells from control and treatment groups were isolated using reported procedures. Briefly, Splenocytes were obtained by crushing the spleen using syringe plunger. The resulting Splenocytes were lysed using (0.15 M NH4Cl, 10 mM NaHCO3, 0.1 mM EDTA, pH 7.3). Splenocytes were cultured at 4×10⁶ cells/well in RPMI 1640 (2 mM L-Glutamine, 10% FCS) for 96 hours in presence of Pertussis antigens in presence and absence of GTL003. The cell supernatants were collected and cytokine levels were analyzed using commercial multiplex bead-based immunoassay kits according to the manufacturer's instructions (Bio-Rad Laboratories, Hercules, Calif.).

1.5. Humoral Immune Response Profiling

Antibody titres and isotyping was done using multiplex bead based immunoassay. Color-coded carboxylated microspheres representing distinct bead regions were obtained from Luminex Laboratories. Purified pertussis antigens (PT toxin, pertactin and FHA) were coupled to distinct activated beads essentially as described by van Gageldonk et al (25). Commercially available kit from Luminex technologies were used to couple the antigens to the beads. The kit is based on activation of beads by EDC and sulfo-NHS chemistry. Phycoerythrin (RPE)-conjugated goat anti-mouse total IgG, IgG1, IgG2a, IgG2b and IgG3 antibodies from Santa cruz were used for the assay. Purified pertussis antigens were obtained from Serum Institute of India Ltd. Humoral responses to GTL003 was analyzed using conventional ELISA. Briefly, optimized concentration of GTL003 was used for coating the plates. The sera samples were added and captured antibodies were detected using anti-mouse enzyme conjugated antibodies. The titre was determined as highest dilution of antibody showing the response higher than the cut-off OD.

1.6. Respiratory Challenge

Six mice each group were inoculated intraperitoneally with 0.5 ml from the test and reference vaccines (Mills et al. 1998, Xing et al. 1999). On day 21, B. pertussis suspension (2×10⁸ CFU/ml) was instilled in each mouse using intranasal challenge. Infected mice were sacrificed 7 days post-challenge. After ablation, the lung of each animal was homogenized and plated on BG and incubated at 37° C. for five days for determination of viable counts (Colony Forming Units—CFU).

1.7 Evaluation of Impact of GTL003 on Tetanus and Diphtheria Antibody Titres in Tdap Vaccines

The experiments were conducted in accordance with the following design:

| | Control group | Test group |
|---|---|---|
| Experimental | Tdap Vaccine alone | Tdap Vaccine + 25 μg |
| groups | | of GTL003 |
| immunization | Day 0, 14, 28 | |
| Bleed on | Day 42 | |
| Number of animals | 10 | |
| per group | | |
| Assay | Titre estimation using | |
| | Luminex assay | |
| Antibody titres | Geometric Mean Titres | |
| reported as | | |

The obtained results were as follows:

| GTL003 in prime | Diphtheria GMT | Tetanus GMT |
|---|---|---|
| and boost | at Day 42 | at Day 42 |
| Tdap Vaccine alone | 19401 | 235253 |
| Tdap Vaccine + | 25401 | 235253 |
| GTL003 | | |

1.8 Effect of GTL003 on Th1 Cytokine (IFN-Gamma) Levels when Formulated with Tdap Vaccine and Administered as Booster Vaccine

In order to assay the effect of repeated administration of Tdap vaccine and Tdap+GTL003 on IFN-gamma levels, the following experiments were carried out:

| | Day 0 | Booster 1 | Booster 2 | | Stimulus | IFNGamma |
|---|---|---|---|---|---|---|
| Route | Immunization | (Day 28) | (Day 56) | Stimulus | time | Median value |
| IM | Tdap (1:10) | | | aP | 96 hrs | 2282 |
| | | | | (10 μg/ml) | | (day 28) |
| IM | Tdap (1:10) | Tdap(1:10) | | aP | 96 hrs | 484 |
| | | | | (10 μg/ml) | | (day 56) |
| IM | Tdap (1:10) | Tdap(1:10) | Tdap(1:10) | aP | 96 hrs | 200 |
| | | | | (10 μg/ml) | | (day 72) |
| GTL003 Treatment | | | | | | |
| groups | | | | | | |
| IM | Tdap (1:10) | Tdap(1:10) + | | aP | 96 hrs | 1900 |
| | | 25 μg | | (10 μg/ml) | | (day 56) |
| | | GTL003 | | | | |
| IM | Tdap (1:10) | Tdap(1:10) + | Tdap(1:10) + | aP | 96 hrs | 1600 |
| | | 25 μg | 25 μg | (10 μg/ml) | | (day 72) |
| | | GTL003 | GTL003 | | | |

Median values are representative of data from 6 animals (spleen processed in 3 different pools of 2 spleen each). Splenocytes were stimulated (4 million cells/well) with mixture of aP antigens (10 μg each) and at 96 hours the supernatants were collected for cytokine analysis.

2. Results

2.1. Mice Immune Response after Prime with Tdap and Boost with Tdap⁺ GTL003

To assess the impact of GTL003 in the booster vaccination on the immune response induced in primed mice, animals were primed with one injection of alum adjuvanted-Tdap at day 0 and received the boost injection containing alum adjuvanted-Tdap with or without GTL003 added to the formulation at day 28. In another group animals were primed with one injection of alum adjuvanted-Tdap at day 0 and received the boost injection containing Whole cell Vaccine without Alum at day 28. Three injection routes where tested: intra-muscular (IM), intra-peritoneal (IP) and subcutaneous (SC).

2.1.1. Cytokine Immune Response: Towards a Th1/Th17 Immune Response

Aluminium hydroxide adjuvanted-Tdap vaccinated people develop a more pronounced Th2 and lower Th1 immune response than induced by wP vaccination. This is thought to be the reason why immunity wanes more rapidly compared to immunity in people vaccinated with the WCV that is more prone to induce a TH1 and TH17 types of immune response (10, 15). (Bancroft)

IM Immunization

Animals primed by IM injection of Tdap/Alum and boosted with Tdap/Alum+GTL003 developed a higher antigen-specific IFN-g immune response as well as a higher antigen-specific IL-17 immune response compared to animals primed with Tdap and boosted with Tdap alone. Ratios were calculated between the level of IL-4 or IL5 vs IFN-g secreted by antigen-stimulated spleen cells to assess the impact of the booster of this TH1/TH2 ratio.

TABLE 3
Cytokine response measured after mice immunization either with Tdap
(prime) + Tdap (boost) or with Tdap (prime)/Tdap + GTL003 (Boost) (pg/ml).
Ratio of IFN-g/IL-4 and IFNg/IL5 secreted cytokines is also calculated.
Splenocytes restimulations were performed with Ap antigens + GTL003

| Group | | | | IFN | | | | | |
|---|---|---|---|---|---|---|---|---|---|
| Prime | Boost | Stimulus | Route | Gamma | IL4 | IL5 | IL-17 | IFNg/IL4 | IFNg/IL5 |
| TdaP | TdaP (1:10) | Ap + | IM | 388 | 210 | 610 | 389 | 1.85 | 0.64 |
| (1:10) | | GTL003 | | | | | | | |
| TdaP | TdaP(1:10) + | Ap + | IM | 1800 | 753 | 278 | 969 | 2.39 | 6.47 |
| (1:10) | GTL003 | GTL003 | | | | | | | |

As shown in Table 3, surprisingly, the addition of GTL003 in the booster skewed the immune response towards a Th1 immune response, i.e. increased IFNg/IL-4 and IFNg/IL-5 ratio compared to the TdaP boost without GTL003. Of note, the IL-17 response was also increased.

When we then compared the effect of GTL003 on the Th1 immune response when added to a Tdap+GTL003 boost vs the WCV boost, the IFNg immune response was higher in animals that have been boosted with Tdap+GTL003 (Table 4) whereas the Il-17 is equivalent.

TABLE 4
Cytokine response measured after mice immunization either with Tdap
(prime) + Tdap (boost) or with Tdap (prime) + WCV (Boost) (pg/ml).
Ratio of IFN-g/IL-4 and IFNg/IL5 secreted cytokines is also calculated.
Splenocytes restimulations were performed with Ap antigens + GTL003.

| Group | | | | | | | | IFNg/ | IFNg/ |
|---|---|---|---|---|---|---|---|---|---|
| Prime | Boost | Stimulus | Route | IFNg | IL4 | IL5 | IL-17 | IL4 | IL5 |
| TdaP | TdaP + | Ap + | IM | 1800 | 753 | 278 | 969 | 2.39 | 6.47 |
| (1:10) | GTL003(1:10) | GTL003 | | | | | | | |
| TdaP | WCV | Ap + | IM | 811 | 435 | 729 | 914 | 1.86 | 1.11 |
| (1:10) | | GTL003 | | | | | | | |

When looking at the IFNg/IL-4 ratio and IFNg/IL-5 ratio, they were more-Th1 oriented response in the Tdap+GTL003 boost compared to the WCV boost.

IP Immunization:

The same immunizations were performed via the intraperitoneal route. Surprisingly, the results were not the same as via the IM route. The Tdap prime/Tdap boost induced a better TH1 profile compared to the Tdap prime/Tdap+GTL003 boost. No skew of the response towards a TH1 is observed when adding GTL003 in the Tdap boost (Table 5) or WCV in the boost (table 6)

TABLE 5
Cytokine response measured after mice immunization either with Tdap
(prime) + Tdap (boost) or with Tdap (prime)/Tdap + GTL003 (Boost) (pg/ml).
Ratio of IFN-g/IL-4 and IFNg/IL5 secreted cytokines was also calculated.
Splenocytes restimulations were performed with Ap antigens + GTL003.

| Group | | | | | | | | | |
|---|---|---|---|---|---|---|---|---|---|
| Prime | Boost | Stimulus | Route | IFNg | IL4 | IL5 | IL-17 | IFNg/IL4 | IFNg/IL5 |
| TdaP | TdaP | Ap + | IP | 838 | 1207 | 1816 | 1810 | 0.69 | 0.46 |
| (1:10) | (1:10) | GTL003 | | | | | | | |
| TdaP | TdaP + | Ap + | IP | 341 | 2207 | 1492 | 209 | 0.15 | 0.23 |
| (1:10) | GTL003 | GTL003 | | | | | | | |
| | (1:10) | | | | | | | | |

Finally, when comparing the WCV boost with Tdap+GTL003 boost via the IP route, WCV boost allowed a slightly better IFNg and IL-17 immune response (Table 6). The overall response was nevertheless Th2 oriented.

TABLE 6
Cytokine response measured after mice immunization either with Tdap
(prime)/Tdap GTL003 (boost) or with Tdap (prime) + WCV (Boost) (pg/ml).
Ratio of IFN-g/IL-4 and IFNg/IL5 secreted cytokines was also calculated.
Splenocytes restimulations were performed with Ap antigens + GTL003.

| Group | | | | | | | | | |
|---|---|---|---|---|---|---|---|---|---|
| Prime | Boost | Stimulus | Route | IFNg | IL4 | IL5 | IL-17 | IFNg/IL4 | IFNg/IL5 |
| TdaP | TdaP + | Ap + | IP | 341 | 2207 | 1492 | 209 | 0.15 | 0.23 |
| (1:10) | GTL003 | GTL003 | | | | | | | |
| | (1:10) | | | | | | | | |
| TdaP | WCV | Ap + | IP | 751 | 827 | 2719 | 1637 | 0.91 | 0.28 |
| (1:10) | | GTL003 | | | | | | | |

SC Immunization:

No significant difference was observed between the Tdap prime/Tdap boost vs Tdap prime/tdap+GTL003 boost in terms of cytokine responses.

2.1.2. Humoral Immune Response

The antibody response to FHA, and PT was measured in the sera of vaccinated animals with Tdap and boosted either with Tdap+GTL003 or WCV.

FHA-Specific Antibodies:

Animals boosted with Tdap+GTL003 had a slight decrease in IgG1 antibodiy response with almost no IgG2a, an increase in IgG2b and IgG3 levels allowing to say that the Ab response is a mixed Th1/Th2 profile but with a trend towards a Th1 IgG isotype response in the presence of GTL003.

Animals boosted with WCV developed a similar pattern of response except that IgG1 Ab level did not change while IgG2b ab level increased dramatically.

PT-Specific Antibodies:

A decrease of Th2 IgG1 isotype response post-Tdap+GTL003 boost was measured. IgG2a Ab were the same and IgG2b and IgG3 Ab decreased. The same pattern of responses was obtained in animals boosted with WCV.

PRN-Specific Antibodies:

The levels of PRN-specific IgG were below the limit of detection of our assays at the dilutions used for all vaccine conditions

GTL003-Specific Antibody:

The anti-GTL003 IgG titers of are significantly higher after boosting with Tdap+GTL003 than with WCV. This is suggesting that the GTL003-containing vaccine boost will provide a better anti-CyaA mediated protection against whooping cough than WCV.

TABLE 7
Serum Anti-Cya antibody levels in animals immunized with GTL003
or WCV. Animals were primed with Tdap

| Route | 1st dose | 2^{nd} dose | GMT |
|---|---|---|---|
| IM | Tdap | Whole cell vaccine | 800 |
| IM | Tdap (1:10) | Tdap (1:10) + GTL003 | 12800 |

2.2. Protection of Mice Vaccinated with Tdap+Gtl003 Compared to wP or Tdap Alone

BalbC mice were vaccinated at day 0, intraperitoneally, with either wP, Tdap alone or Tdap with increasing doses of GTL003 (10 μg, 25 μg, 50 μg and 100 μg). Three dilutions were tested as shown in Table 8. Animals were challenged 21 days after vaccination with Bordetella pertussis strain. Seven days after challenge, lungs were analyzed for the presence of pertussis colonization.

TABLE 8
Intranasal Challenge assay post vaccination
Lung CFU distribution

| | | | |
|---|---|---|---|
| Vaccine | 2 | 10 | 50 |
| dilution | | | |
| Whole Cell | 508 | 4383 | 86000 |
| vaccine | | | |
| Tdap (Control | 188 | 16931 | 62167 |
| group) | | | |
| Tdap + GTL003 | 37 | 52 | 1935 |
| (10 μg) | | | |

Tdap+GTL003 vaccine induces overall a better protection against B. pertussis than Tdap alone and equivalent or better than WCV. For vaccine dilution 10, protection was observed with Tdap+10 μg GTL003 but also 25 (1028 CFU) and 50 μg (2625 CFU) GTL003 compared to Tdap alone and WCV.

Analysis of the immune response after a single intra-peritoneal injection of Tdap+GTL003 was performed to understand the protection gain in the presence of GTL003.

2.3. Mice Immune Response after Single Dose Vaccination with Tdap+GTL003

2.3.1 Humoral Immune Response

Four groups of mice were vaccinated either with the Aluminium hydroxide adjuvanted-Tdap alone or adjuvanted-Tdap mixed with increasing doses of GTL003 as presented in the table hereafter.

TABLE 9
Serum anti-aP antibody levels in vaccinated mice

| Vaccine composition | | Total IgG titers | | |
|---|---|---|---|---|
| | GTL003 | Anti-PT | Anti-FHA | Anti-PRN |
| Vaccinated Groups | μg | Ab | Ab | Ab |
| Tdap (ref) | 0 | 49.2 | 117.2 | 6.7 |
| Tdap + GTL003 | 4 | 79.7 | 127 | 10.54 |
| Tdap + GTL003 | 40 | 96.8 | 142.9 | 14.38 |
| Tdap + GTL003 | 120 | 73 | 194.2 | 5.03 |

A slight increase of antibodies against PT, FHA and PRN was detected only in groups of mice immunized with pertussis vaccine containing GTL003.

When comparing the production of antibodies against CyaA, the response was higher with adjuvanted-Tdap+GTL003 vaccinated animals compared with those vaccinated with the WCV. Of note, WCV contains wild type CyaA.

TABLE 10
Serum anti-CyaA antibody levels in vaccinated mice.

| | | | Anti-cyaA |
|---|---|---|---|
| Route | Prime | Boost | IgG Titer |
| IP | Tdap (1:10) | Whole cell vaccine | 800 |
| IP | Tdap (1:10) | Tdap (1:10) + | 12800 |
| | | GTL003 | |

2.3.2 Cytokine Production by Spleen and Macrophages

The secreted cytokines by spleen cells restimulated in vitro with Ap antigens from mice vaccinated with either adjuvanted-Tdap+GTL003 or Tdap alone or WCV were analyzed in unchallenged mice, i.e. mice that have not been challenged in vivo by live B. pertussis post vaccination.

TABLE 11
Cytokine immune response measured in spleens and macrophages of vaccinated, but
unchallenged mice, after in vitro stimulation with vaccine antigens.

| | Groups according to GTL003 dose (10, 25, 50, 100 μg) injected IP with | | | | |
|---|---|---|---|---|---|
| | Tdap +/− GTL003 or WCV (Tdap + GTL003 dose in μg) | | | | |
| Readout | Tdap | Tdap + 25 | Tdap + 50 | Tdap + 100 | WCV |

Th1 cytokines

| | | | | | |
|---|---|---|---|---|---|
| IL-12 | Reference | No | No | No | − |
| (p70) | | change | change | change | |
| IFN-g | Reference | + | − | − | − |

Th2 cytokines

| | | | | | |
|---|---|---|---|---|---|
| IL-4 | Reference | − | − | − | NA |
| IL-5 | Reference | No | No | − | NA |
| | | change | change | | |
| IL-13 | Reference | No | + | No | − |
| | | change | | change | |
| IL-9 | Reference | No | − | − | No |
| | | change | | | change |

Th17 cytokines

| | | | | | |
|---|---|---|---|---|---|
| IL-17A | Reference | ++ | + | ++ | NA |

Macrophage activity

| | | | | | |
|---|---|---|---|---|---|
| Nitric | Reference | ++ | +++ | + | NA |
| Oxide | | | | | |

+, ++, +++: 1-2×, 2-4× and >4× increase in cytokine level compared to cytokine induced by Tdap alone (reference), respectively;
−: decrease

The addition of GTL003 at the concentration of either 25 or 50 μg to the Tdap vaccine induces an increase of the TH1 and TH17 immune responses. Unexpectedly, GTL003 induces the same antigen-specific effect on nitric oxide (NO) in stimulated spleen cells while the injection has been performed intra-peritonealy compared to mice vaccinated with Tdap alone. In previous experiments, NO has been shown to increase after peritoneal macrophages stimulation after intra-peritoneal injection that could be expected because of the recruitment of macrophages at injection site. The same conclusions on the effect of the addition of GTL003 to Tdap vaccine are drawn in vaccinated mice followed by an intranasal challenge with live B. pertussis.

TABLE 12
Analysis of the cytokine profile after challenge
of mice vaccinated with adjuvanted-Tdap, adjuvanted
Tdap + GTL003 or WCV. Tdap + GTL003 dose in μg

| | Groups according to GTL003 dose (10, 25, 50, 100) | | | | |
|---|---|---|---|---|---|
| | groups injected IP with Tdap +/− GTL003 or WCV | | | | |
| | | Tdap + | Tdap + | Tdap + | |
| Readout | Tdap | 25 | 50 | 100 | WCV |

Th1 cytokines

| | | | | | | |
|---|---|---|---|---|---|---|
| IL-12 (p70) | Reference | ++ | ++ | ++ | = | ++ |
| IFN-gamma | Reference | +++ | +++ | +++ | = | +++ |

Th2 cytokines

| | | | | | | |
|---|---|---|---|---|---|---|
| IL-4 | Reference | + | +++ | +++ | ≥ | + |
| IL-5 | Reference | +++ | +++ | +++ | = | +++ |
| IL-13 | Reference | ++ | +++ | +++ | ≥ | ++ |
| IL-9 | Reference | + | + | + | > | No |
| | | | | | | change |

Th17 cytokines

| | | | | | | |
|---|---|---|---|---|---|---|
| IL-17A | Reference | ++ | +++ | +++ | ≥ | ++ |

Pro-inflammatory cytokines

| | | | | | | |
|---|---|---|---|---|---|---|
| IL-1alpha | Reference | ++ | +++ | ++ | ≥ | + |
| IL-1beta | Reference | + | + | + | = | + |
| IL-6 | Reference | +++ | +++ | +++ | = | +++ |
| TNFalpha | Reference | + | + | + | = | + |

Macrophage activity

| | | | | | | |
|---|---|---|---|---|---|---|
| Nitric | NA | ++ | − | − | > | Reference |
| Oxide | | | | | | |

+,
++,
+++: 1-2x, 2-4x and >4x increase in cytokine level compared to cytokine induced by Tdap alone (reference), respectively;
−: decrease

A dramatic overall increase in the immune response in the groups vaccinated with GTL003-containing adjuvanted-Tdap+GTL003 compared to adjuvanted Tdap without GTL003 was observed. This response was equivalent or superior to WCV. A strong increase of IFN-g, IL17 and Nitric Oxide were observed.

The analysis of the IFN-g after stimulation either with Tdap antigens or Tdap+GTL003 showed that the response against all antigens was increased in the groups immunized with GTL003-containing vaccine.

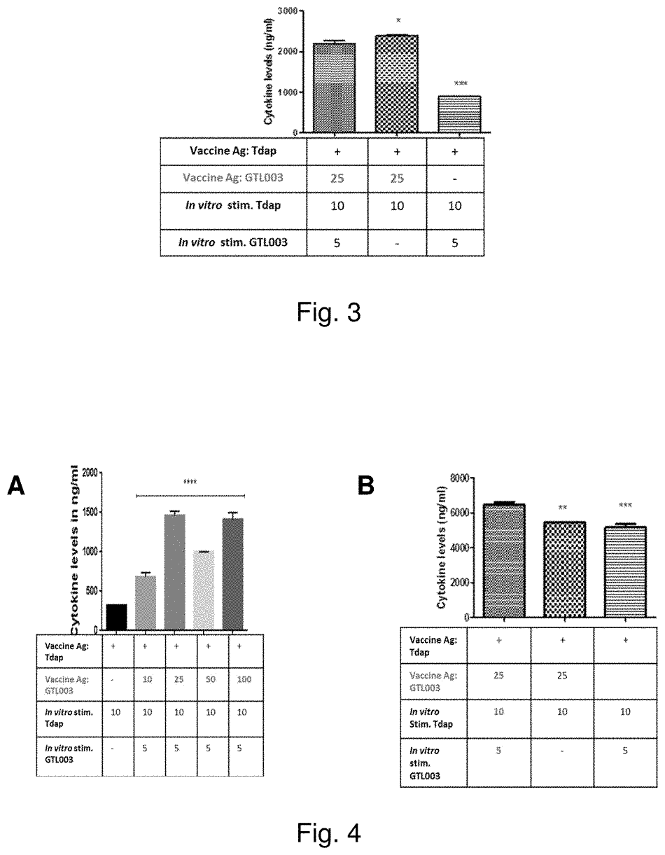

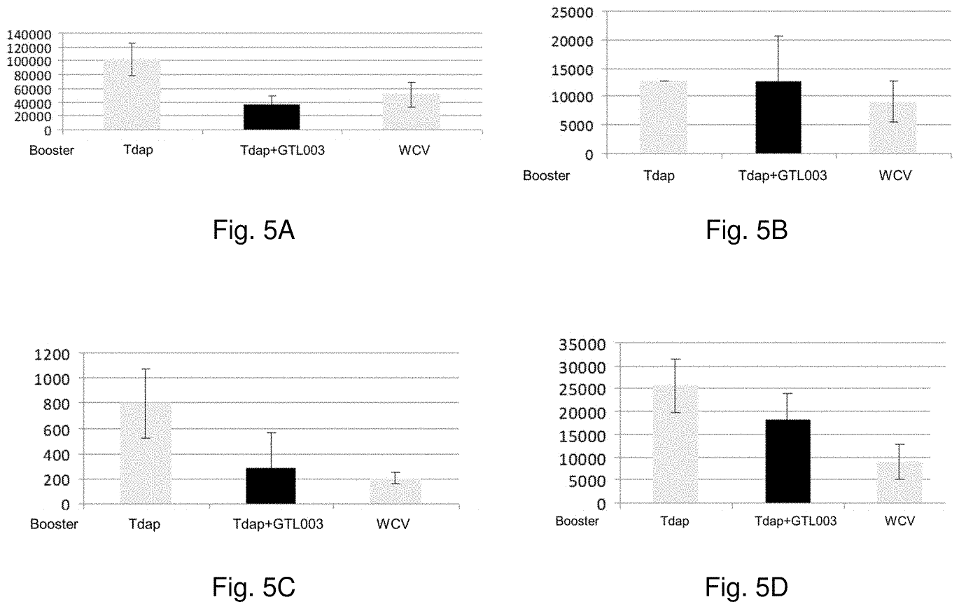

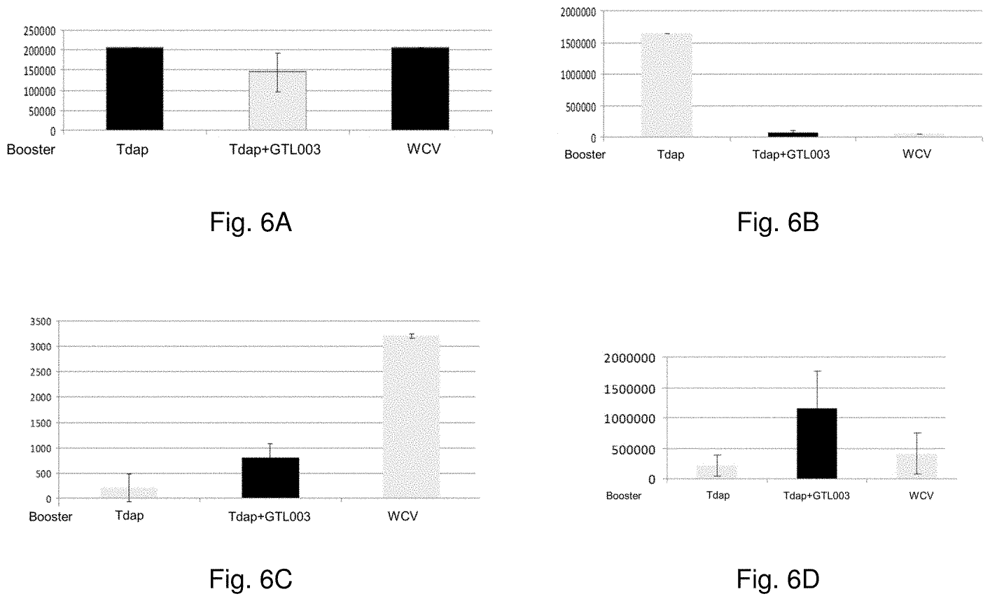

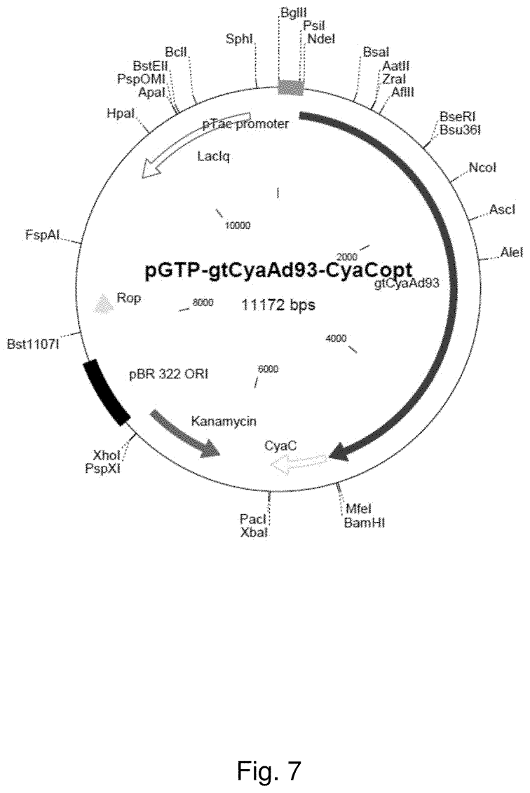

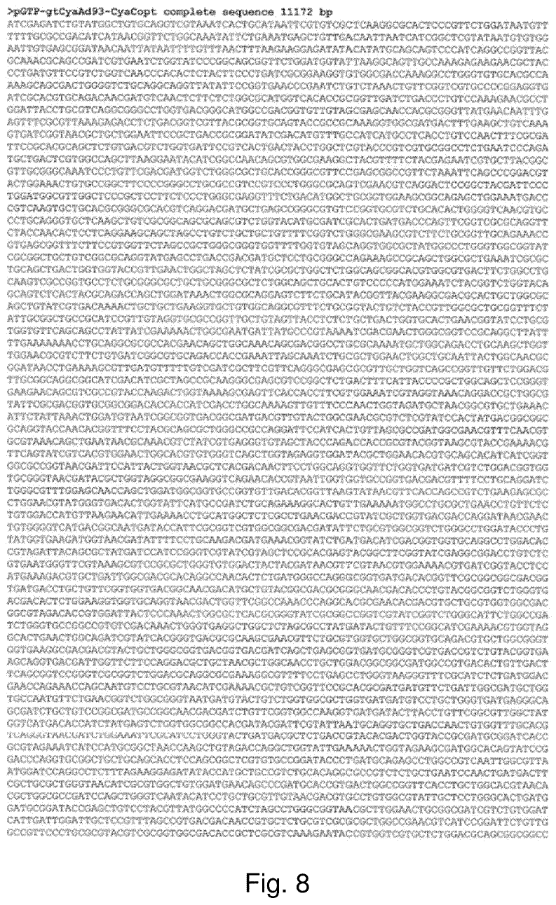

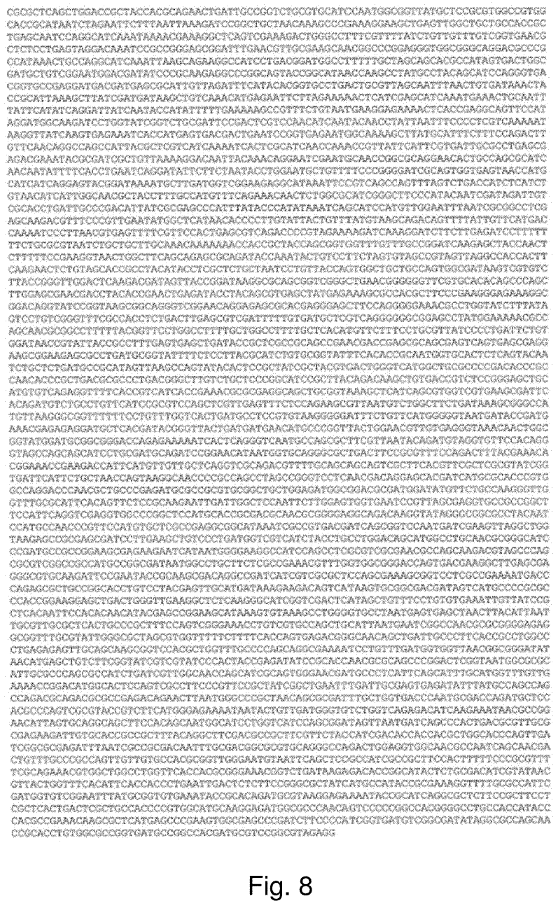

For IL-17, the level of cytokine measured was higher in the presence of GTL003 and increased between 10 and 25 μg of GTL003 (FIG. 3A). Unexpectedly, the increased response in those animals vaccinated with Tdap+GTL003 was GTL003-specific (FIG. 4 B).

### Conclusion

These results show for the first time the ability of GTL003 to enhance the Th1 immune response against pertussis antigens when included in a Tdap vaccine containing aluminium, a Th2 adjuvant.
The effect is observed after intramuscular injection when GTL003 is in the Tdap boost which is unexpected following what the literature reports;
The effect is observed after a single intraperitoneal injection (standard route of vaccination in a challenge assay) allowing a better protection of animals, which is supported by the cytokine and humoral responses that are superior to the WCV; this is also unexpected in the presence of aluminium.

2.4 Evaluation of the Impact of GTL003 on Tetanus and Diphtheria Antibody Titres in Tdap Vaccine

From the obtained data, no interfering effect of GTL003 on Tetanus and Diphtheria antibody titres was observed. Titres of GTL003 treatment groups were similar to titres observed in control Tdap Vaccine.

2.5 Effect of GTL003 on Th1 Cytokine (IFN-Gamma) Levels when Formulated with Tdap Vaccine and Administered as Booster Vaccine

Administration of multiple doses of Tdap results in excessive Th2-bias wherein a significant reduction of IFN-gamma levels was noted. By contrast, administration of GTL003 with Tdap at the time of boosting prevented a significant drop in IFN-gamma levels in treatment groups even when the number of doses of Tdap increases. Accordingly, this confirms that the administration of GTL003 in the booster dose of vaccine skews the response toward a Th1 response.

### REFERENCES

1. Cherry, J. D. 1996. Historical review of pertussis and the classical vaccine. J Infect Dis 174 Suppl 3:S259-263.
2. Mattoo, S., and J. D. Cherry. 2005. Molecular pathogenesis, epidemiology, and clinical manifestations of respiratory infections due to Bordetella pertussis and other Bordetella subspecies. Clin Microbiol Rev 18:326-382.
3. Aoyama, T., Y. Murase, T. Kato, and T. Iwata. 1985. Efficacy of an acellular pertussis vaccine in Japan. J Pediatr 107:180-183.
4. 1992. American Academy of Pediatrics Committee on Infectious Disease: Acellular pertussis vaccines: recommendations for use as the fourth and fifth doses. Pediatrics 90:121-123.
5. Sealey, K. L., T. Belcher, and A. Preston. 2016. Bordetella pertussis epidemiology and evolution in the light of pertussis resurgence. Infect Genet Evol 40:136-143.
6. Edwards, K. M., and G. A. Berbers. 2014. Immune responses to pertussis vaccines and disease. J Infect Dis 209 Suppl 1:S10-15.
7. Allen, A. 2013. Public health. The pertussis paradox. Science 341:454-455.
8. Cherry, J. D. 2012. Epidemic pertussis in 2012—the resurgence of a vaccine-preventable disease. N Engl J Med 367:785-787.
9. Black, R. E., S. Cousens, H. L. Johnson, J. E. Lawn, I. Rudan, D. G. Bassani, P. Jha, H. Campbell, C. F. Walker, R. Cibulskis, T. Eisele, L. Liu, and C. Mathers. 2010. Global, regional, and national causes of child mortality in 2008: a systematic analysis. Lancet 375:1969-1987.
10. Warfel, J. M., and K. M. Edwards. 2015. Pertussis vaccines and the challenge of inducing durable immunity. Curr Opin Immunol 35:48-54.
11. Witt, M. A., L. Arias, P. H. Katz, E. T. Truong, and D. J. Witt. 2013. Reduced risk of pertussis among persons ever vaccinated with whole cell pertussis vaccine compared to recipients of acellular pertussis vaccines in a large US cohort. Clin Infect Dis 56:1248-1254.
12. Klein, N. P., J. Bartlett, A. Rowhani-Rahbar, B. Fireman, and R. Baxter. 2012. Waning protection after fifth dose of acellular pertussis vaccine in children. N Engl J Med 367:1012-1019.
13. Koepke, R., J. C. Eickhoff, R. A. Ayele, A. B. Petit, S. L. Schauer, D. J. Hopfensperger, J. H. Conway, and J. P. Davis. 2014. Estimating the effectiveness of tetanus-diphtheria-acellular pertussis vaccine (Tdap) for preventing pertussis: evidence of rapidly waning immunity and difference in effectiveness by Tdap brand. J Infect Dis 210:942-953.
14. Warfel, J. M., L. I. Zimmerman, and T. J. Merkel. 2014. Acellular pertussis vaccines protect against disease but fail to prevent infection and transmission in a nonhuman primate model. Proc Natl Acad Sci USA 111:787-792.
15. Higgins, S. C., A. G. Jarnicki, E. C. Lavelle, and K. H. Mills. 2006. TLR4 mediates vaccine-induced protective cellular immunity to Bordetella pertussis: role of IL-17-producing T cells. J Immunol 177:7980-7989.
16. Guermonprez, P., N. Khelef, E. Blouin, P. Rieu, P. Ricciardi-Castagnoli, N. Guiso, D. Ladant, and C. Leclerc. 2001. The adenylate cyclase toxin of Bordetella pertussis binds to target cells via the alpha(M)beta(2) integrin (CD11b/CD18). J Exp Med 193:1035-1044.
17. Hewlett, E. L., L. Gray, M. Allietta, I. Ehrmann, V. M. Gordon, and M. C. Gray. 1991. Adenylate cyclase toxin from Bordetella pertussis. Conformational change associated with toxin activity. J Biol Chem 266:17503-17508.
18. Vojtova, J., J. Kamanova, and P. Sebo. 2006. Bordetella adenylate cyclase toxin: a swift saboteur of host defense. Curr Opin Microbiol 9:69-75.
19. Goodwin, M. S., and A. A. Weiss. 1990. Adenylate cyclase toxin is critical for colonization and pertussis toxin is critical for lethal infection by Bordetella pertussis in infant mice. Infect Immun 58:3445-3447.
20. Khelef, N., H. Sakamoto, and N. Guiso. 1992. Both adenylate cyclase and hemolytic activities are required by Bordetella pertussis to initiate infection. Microb Pathog 12:227-235.
21. Macdonald-Fyall, J., D. Xing, M. Corbel, S. Baillie, R. Parton, and J. Coote. 2004. Adjuvanticity of native and detoxified adenylate cyclase toxin of Bordetella pertussis towards co-administered antigens. Vaccine 22:4270-4281.
22. Cheung, G. Y., D. Xing, S. Prior, M. J. Corbel, R. Parton, and J. G. Coote. 2006. Effect of different forms of adenylate cyclase toxin of Bordetella pertussis on protection afforded by an acellular pertussis vaccine in a murine model. Infect Immun 74:6797-6805.
23. Dunne, A., P. J. Ross, E. Pospisilova, J. Masin, A. Meaney, C. E. Sutton, Y. Iwakura, J. Tschopp, P. Sebo, and K. H. Mills. 2010. Inflammasome activation by adenylate cyclase toxin directs Th17 responses and protection against Bordetella pertussis. J Immunol 185:1711-1719.
24. Dadaglio, G., C. Fayolle, X. Zhang, B. Ryffel, M. Oberkampf, T. Felix, S. Hervas-Stubbs, R. Osicka, P. Sebo, D. Ladant, and C. Leclerc. 2014. Antigen targeting to CD11b+ dendritic cells in association with TLR4/TRIF signaling promotes strong CD8+ T cell responses. J Immunol 193:1787-1798.
25. van Gageldonk P. G., van Schaijk F. G., van der Klis F. R., Berbers G. A. 2008. Development and validation of a multiplex immunoassay for the simultaneous determination of serum antibodies to Bordetella pertussis, diphtheria and tetanus. J. Immunol. Methods 335:79-89
26. Bancroft et al, Cellular Immunology, 2016
27. Ryan et al, Clin Exp Immunol 2000

1. An immunogenic composition which is chosen in the group of:
a) an acellular immunogenic composition suitable for the protection against a condition causally related to the infection by a Bordetella strain, in particular Bordetella pertussis in a human host, or
b) a combination immunogenic composition comprising said acellular immunogenic composition suitable for the protection against a condition causally related to the infection by a Bordetella strain, in particular Bordetella pertussis in a human host
wherein said acellular immunogenic composition comprises:
a non-toxic polypeptide, in particular a non-cytotoxic polypeptide, derived from the adenylate cyclase protein (CyaA-derived polypeptide) of a Bordetella strain and,
optionally an adjuvant of the TH1 immune response or an adjuvant of the TH2 immune response or a combination of both.

2. An immunogenic composition according to claim 1, wherein the non-cytotoxic polypeptide, derived from the adenylate cyclase protein (CyaA-derived polypeptide) of a Bordetella strain is (i) a polypeptide the amino acid sequence of which is obtained from CyaA of Bordetella pertussis and contains a deletion of a continuous segment of at least 93 amino acid residues from position 227 to position 321 in the amino acid sequence of the native CyaA of Bordetella pertussis or (ii) a polypeptide which is a variant of the polypeptide in (i) the amino acid sequence of said variant polypeptide being obtained from CyaA of Bordetella parapertussis, Bordetella bronchiseptica or Bordetella hinzii, and containing the deletion of a continuous segment of at least 93 amino acid residues defined by the positions in the native CyaA sequence of Bordetella parapertussis, Bordetella bronchiseptica or Bordetella hinzii matching positions 227 and 321 in CyaA of said continuous segment of at least 93 amino acid residues of Bordetella pertussis.

3. An acellular immunogenic composition according to claim 1 or 2 suitable for the protection against a condition causally related to the infection by a Bordetella strain, in particular Bordetella pertussis, in a human which comprises:
at least two antigens from a Bordetella strain wherein one antigen is (i) a non-toxic polypeptide, in particular a non-cytotoxic polypeptide, derived from the adenylcyclase protein (CyaA-derived polypeptide) of a Bordetella strain, said polypeptide being in particular (i.1) either a polypeptide the amino acid sequence of which is obtained from CyaA of Bordetella pertussis and contains a deletion of a continuous segment of at least 93 amino acid residues from position 227 to position 321 in the amino acid sequence of the native CyaA of Bordetella pertussis, (i.2) or a polypeptide which is a variant of the polypeptide in (i) the amino acid sequence of said variant polypeptide being obtained from CyaA of Bordetella parapertussis, Bordetella bronchiseptica or Bordetella hinzii, and containing the deletion of a continuous segment of at least 93 amino acid residues defined by the positions in the native CyaA sequence of Bordetella parapertussis, Bordetella bronchiseptica or Bordetella hinzii matching positions 227 and 321 in CyaA of said continuous segment of at least 93 amino acid residues of Bordetella pertussis, and wherein further antigen(s) is (are) selected in the group of (ii) Pertussis toxoid (PT), (iii) Filamentous Haemagglutinin (FHA), (iv) Pertactin (PRN) and (v) Fimbriae (Fim),
wherein a protective immune response against the Bordetella strain is raised against said antigens and,
optionally an adjuvant of the TH1 immune response or an adjuvant of the TH2 immune response or a combination of both.

4. A Combination immunogenic composition according to claim 1 or to claim 2, comprising further antigens as active ingredients for the elicitation of an immune protection against determined pathogens which are (vi) at least one antigen of Clostridium tetani consisting of the Tetanus toxin in detoxified form, (vii) an antigen of Corynebacterium complex consisting of the diphtheria toxin in detoxified from, and optionally (viii) further antigens of different pathogen(s).

5. A combination immunogenic composition according to any one of claims 1 to 4 which comprises further antigens as active ingredients for the elicitation of an immune protection against determined pathogens which are selected in the group of Hepatitis B surface antigen (HBs), inactivated poliovirus (IPV) of one or several virus strains, Haemophilus influenza type b polysaccharide.

6. An acellular immunogenic composition according to any one of claims 1 to 3 or a combination immunogenic composition according to any one of claim 1, 4 or 5, wherein the antigens selected in the group of Bordetella toxin in detoxified form (PT), Filamentous Haemagglutinin (FHA), Pertactin (PRN) and Fimbriae (Fim) are independently of each other from Bordetella pertussis, Bordetella parapertussis, Bordetella bronchiseptica or Bordetella hinzii, preferably from B. pertussis, and preferably are all from the same Bordetella strain.

7. Combination immunogenic composition according to any one of claims 1, 2 or 4 to 5 wherein one dose of 0.5 ml of the immunogenic composition contains:
Bordetella toxin in detoxified form, in particular Pertussis toxoid: 1 to 50 micrograms;
Filamentous Haemagglutinin: 1 to 50 μg;
Pertactin: 1 to 20 μg;
optionally Fimbriae: 2 to 25 μg;
Tetanus toxoid: at least 0.25 and less than 25 Lf;
Diphtheria toxoid: at least 0.25 and less than 50 Lf;
and optionally,
Hepatitis B surface antigen: 5 μg to 25 μg
inactivated poliovirus: from 5 to 45, in particular from 8 to 40 D-antigen unit per strain;
Haemophilus influenza type b polysaccharide: 1 to 20 μg.

8. Combination immunogenic composition according to any one of claims 1, 2 or 4 to 6, wherein one dose of 0.5 ml of the immunogenic composition comprises:
Bordetella toxin in detoxified form in particular Pertusssis toxoid: 8 μg;
Filamentous Haemagglutinin: 8 μg;
Pertactin: 2.5 μg;
Tetanus toxoid: 5 Lf;
Diphteria toxoid: 2.5 Lf.

9. An immunogenic composition, in particular a combination immunogenic composition, according to any one of claims 1 to 8 wherein one dose of the immunogenic composition comprises 2.5 to 600 micrograms, 2.5 to 500 μg, 2.5 to 400 μg, 2.5 to 300 μg, 2.5 to 200 μg or 2.5 to 100 μg and preferably comprises 25 μg of the CyaA-derived polypeptide of Bordetella pertussis.

10. An immunogenic composition, in particular a combination immunogenic composition, according to any one of claims 1 to 9 wherein the adjuvant of the TH2 response is an aluminum salt, in particular is aluminum hydroxide, aluminum hydroxyphosphate sulfate or aluminum phosphate.

11. An immunogenic composition, in particular a combination immunogenic composition according to any one of claims 1 to 10 wherein the adjuvant of the TH1 response is selected:
i. in the group of MPL-containing adjuvant, in particular AS15, AS01B, AS01D, or AS01E; CpG-containing adjuvant, in particular AS15, QS21-containing adjuvant, in particular AS01B, D, and E or AS15, immune stimulating complexes (ISCOMs), IC31-containing adjuvant, chitosan-containing adjuvant, liposomal formulation-based adjuvant, in particular AS01B D, E or AS15 or ISCOMs, lipid-based emulsions such as MF59, or
ii. In the group of adjuvants which are or contain a ligand of a toll-like receptor selected among:
a ligand of toll-like receptor 4 (TLR-4), in particular monophosphoryl lipid A (MPL) or Glucopyranosyl Lipid A (GLA) or,
a ligand of toll-like receptor 9 (TLR-9), in particular a synthetic oligodeoxynucleotides (ODNs) containing unmethylated CpG motifs and more particularly HBsAg-1018 or,
a ligand of toll-like receptor 3 (TLR-3), in particular a synthetic analog of double-stranded RNA (dsRNA) and more particularly a composition containing Poly(I:C) such as polyinosinic-polycytidylic acid plus poly-L-lysine double-stranded RNA in the presence of carboxymethylcellulose (Poly (ICLC). or,
a ligand of toll-like receptor 2 (TLR-2), in particular synthetic lipopeptides or a recombinant lipoprotein and,
a ligand of toll-like receptor 5 (TLR-5), in particular a recombinant bacterial flagellin.

12. An immunogenic composition, in particular a combination immunogenic composition, according to any one of claims 1 to 11 wherein the CyaA-derived polypeptide of Bordetella consists of:
1) a segment or a fragment of Bordetella pertussis CyaA protein as set forth in SEQ ID No. 2, the sequence of said segment or fragment beginning with the first residue of SEQ ID No.2 and ending with a residue located from position 183 to position 227 of SEQ ID No.2 or a polypeptide variant obtained from Bordetella parapertussis, Bordetella bronchiseptica or Bordetella hinzii and having at least 95% identity in amino acid residues with the segment or fragment consisting of residues 1 to 183 or 1 to 227 in SEQ ID No.2, fused to
2) a segment or a fragment of Bordetella pertussis CyaA protein as set forth in SEQ ID No. 2, the sequence of said segment or fragment beginning with a residue located from position 321 to position 387 of SEQ ID No.2 and ending with the last residue of SEQ ID No.2 or a polypeptide variant obtained from Bordetella parapertussis, Bordetella bronchiseptica or Bordetella hinzii, and having at least 95% identity in amino acid residues with the segment or fragment consisting of residues 321 to final residue or 387 to final residue in SEQ ID No.2.

13. An immunogenic composition, in particular a combination immunogenic composition according to any one of claims 1 to 12 wherein the CyaA-derived polypeptide of Bordetella consists of:
1) a polypeptide comprising or consisting of the sequence as set forth in SEQ ID NO:10 or a polypeptide variant obtained from Bordetella parapertussis, Bordetella bronchiseptica or Bordetella hinzii and characterized by an amino acid sequence matching the amino acid residue positions of SEQ ID No.10;
2) a polypeptide comprising or consisting of the sequence as set forth in SEQ ID NO:12 or a variant obtained from Bordetella parapertussis, Bordetella bronchiseptica or Bordetella hinzii, and characterized by an amino acid sequence matching the amino acid residue positions of SEQ ID No.12,
3) a polypeptide comprising or consisting of the sequence as set forth in SEQ ID NO:19 or a variant obtained from Bordetella parapertussis, Bordetella bronchiseptica or Bordetella hinzii, and characterized by an amino acid sequence matching the amino acid residue positions of SEQ ID No.19,
4) a polypeptide comprising or consisting of the sequence as set forth in SEQ ID NO:20 or a variant obtained from Bordetella parapertussis, Bordetella bronchiseptica or Bordetella hinzii and characterized by an amino acid sequence matching the amino acid residue positions of SEQ ID No.20.

14. An immunogenic composition, in particular a combination immunogenic composition according to any one of claims 1 to 13, wherein the CyaA-derived polypeptide is the expression product in E. coli cells of the nucleic acid molecule gtCyaad93-CyaC-opt borne by plasmid pGTP-gtCyaad93-CyaC-opt of SEQ ID No. 25.

15. A medicinal composition for administration to a human host which comprises an immunogenic composition according to any one of claims 1 to 14 and which is provided as an administration form selected among a powder, a powder and solution for reconstitution, a liquid, in particular a suspension or a solution, a lyophilized component and a combination of such administration forms.

16. A medicinal composition according to claim 15 wherein one dose for administration to a human host contains 0.25 ml to 1 ml of liquid, in particular of reconstituted product.

17. A medicinal composition according to claim 15 or 16, for administration to a human host which is provided as a pharmaceutical form formulated for administration by injection, in particular for intramuscular injection.

18. A method for preparing a vaccine comprising as active ingredients for protection against determined pathogens or condition causally related to such pathogens, antigens of said pathogens and furthermore a polypeptide derived from the adenylate cyclase protein (CyaA-derived polypeptide) of a Bordetella strain as defined in any of claim 1 or 13, wherein the method comprises the step(s) of:
a) providing antigens selected in the group of (i) at least one of the antigens from Bordetella selected in the group of Pertussis toxin in detoxified form (PT), Filamentous Haemagglutinin (FHA), Pertactin (PRN) and Fimbriae (Fim) and preferably the first three antigens or all of these antigens, (ii) at least one antigen of Clostridium tetani consisting of the Tetanus toxin in detoxified form, (iii) an antigen of Corynebacterium complex consisting of the diphtheria toxin in detoxified form, and (iv) the CyaA-derived polypeptide as defined in any one of claim 1 or 13 to 15 and optionally (v) further antigens of different pathogen(s) as active ingredients for the elicitation of an immune response against said determined pathogens formulated as one or several component(s) and,
b) admixing said component(s) with one or more compounds that enhance the immune response when the vaccine is administered to a host such as adjuvant(s) of the TH2-oriented immune response, adjuvant of the TH1-oriented immune response, adjuvant of the TH17-oriented immune response or a combination of such adjuvants,
wherein steps a) and b) are optionally carried out as a single step.

19. A method according to claim 18 wherein the active ingredients for protection are as defined in any one of claims 2 to 14.

20. A combination immunogenic composition according to any one of claims 1 to 14 or a medicinal composition according to any one of claims 15 to 17 for use in active immunization of a human host against diphtheria, tetanus and condition causally related to pertussis infection, and optionally against hepatitis B, poliomyelitis and/or disease caused by Haemophilus influenza type b.

21. A combination immunogenic composition according to any one of claims 1 to 13 or a medicinal composition according to any one of claims 14 to 16 for use in active immunization of a human host against the persistence of Pertussis bacteria after infection of a human host, or against the transmission and/or against the colonization of airways of the human host infected by a Bordetella strain, in particular B. pertussis.

22. A combination immunogenic composition according to any one of claims 1 to 13 or a medicinal composition according to any one of claims 15 to 17 for use according to claim 19 or 20 as a dose for administration after a first dose, in particular (i) as a second or as a further dose of a multiple-dose setting or (ii) as a booster dose, in individuals who previously received first vaccination dose or primary vaccination with a monovalent or a combination vaccine against at least one of the diseases selected in the group of tetanus, diphtheria and a condition causally related to infection by a Bordetella strain, in particular Bordetella pertussis, and optionally hepatitis B, poliomyelitis and disease caused by Haemophilus influenza type b.

23. A combination immunogenic composition according to any one of claims 1 to 14 or a medicinal composition according to any one of claims 16 to 18 for use according to any one of claims 20 to 22 as a dose for administration as a second or as a further dose, in particular as a booster dose and wherein said second, further, or in particular booster dose, is different from the first or from the previously administered dose(s) in that said first or previous doses is(are) devoid of non-toxic CyaA derived polypeptide as defined in claims 1, 2 or 12 to 14, and preferably is (are) devoid of any CyaA polypeptide or CyaA-derived polypeptide.

24. A combination immunogenic composition or a medicinal composition for use according to any one of claims 20 to 23, wherein the host is a child at birth or later, in particular until 18 months of age

25. A combination immunogenic composition for use according to claim 22 or 23, as a booster dose in a prime/boost vaccination setting wherein the host is a Child over 4 years of age, an adolescent over 11 or an adult, in particular an elderly person, a pregnant woman or relatives close to a pregnant woman, said host having previously received primary vaccination with a different acellular vaccine against a condition causally related to infection by Bordetella strain, in particular Bordetella pertussis either administered as a monovalent pertussis vaccine or a combination vaccine against whopping cough.
